(19) United States
(12) Patent Application Publication
(10) Pub. No.: US 2023/0051500 A1
(43) Pub. Date: Feb. 16, 2023

(54) ENDOSCOPE ILLUMINATION SYSTEM AND ENDOSCOPE USING THE SAME

(71) Applicant: OLYMPUS CORPORATION, Tokyo (JP)

(72) Inventor: Yuki NAGATA, Tokyo (JP)

(73) Assignee: OLYMPUS CORPORATION, Tokyo (JP)

(21) Appl. No.: 17/876,308

(22) Filed: Jul. 28, 2022

(30) Foreign Application Priority Data
Aug. 6, 2021 (JP) PCT/JP2021/029398

(51) Int. Cl. A61B 1/00; A61B 1/06

(52) U.S. Cl. CPC A61B 1/00096; A61B 1/06; G02B 23/2469

(57) ABSTRACT

An endoscope illumination system is provided in an insert part and includes an exit surface from which illumination light is emitted, an entrance side optical surface on which the illumination light is incident, and an exit side optical surface from which the illumination light goes out. The entrance side optical surface includes an inner light distribution surface and an outer light distribution surface. The outer light distribution surface is located farther from the center axis of the insert part than the inner light distribution surface. The inner light distribution surface includes a first inner surface. The first inner surface is a curved surface that is convex to the exit surface. The outer light distribution surface is a flat surface or a curved surface that is concave to the exit surface.

### CROSS REFERENCE TO RELATED APPLICATION

The present application is based upon and claims the benefit of priority from the prior international application No. PCT/JP2021/029398 filed on Aug. 6, 2021; the entire contents of which are incorporated herein by reference.

### BACKGROUND OF INVENTION

### Technical Field

The present invention relates to an endoscope illumination system and an endoscope using the same.

### Description of the Related Art

An illumination optical system for an endoscope is disclosed in Japanese Patent Application Laid-Open No. 2014-054369. The illumination optical system has a light distribution member and a light guide. The light distribution member includes a convex lens. The convex surface of the convex lens is opposed to the exit surface of the light guide. The illumination optical system is provided around an observation optical system.

### SUMMARY OF THE INVENTION

An endoscope illumination system according to at least some embodiments is provided in an insert part and comprises:

an exit surface from which illumination light is emitted;

an entrance side optical surface on which the illumination light is incident; and

an exit side optical surface from which the illumination light goes out,

wherein the entrance side optical surface includes an inner light distribution surface and an outer light distribution surface,

the outer light distribution surface is located farther from the center axis of the insert part than the inner light distribution surface,

the inner light distribution surface includes a first inner surface,

the first inner surface is a curved surface that is convex to the exit surface, and

the outer light distribution surface is a flat surface or a curved surface that is concave to the exit surface.

An endoscope illumination system according to at least some embodiments is provided in an insert part and comprises:

an exit surface from which illumination light is emitted;

an entrance side optical surface on which the illumination light is incident; and

an exit side optical surface from which the illumination light goes out,

wherein the entrance side optical surface includes, in order away from the center axis of the insert part, a first flat surface, a first curved surface abutting on the first flat surface, and a second flat surface abutting on the first curved surface,

the exit side optical surface includes, in order away from the center axis of the insert part, a third flat surface and a second curved surface abutting on the third flat surface,

the first curved surface is a surface convex to the exit surface, and

the second curved surface is a surface convex to the outside.

An endoscope according to at least some embodiments comprises:

the above-described endoscope illumination system; and

an objective optical system, wherein the endoscope illumination system is located farther from the center axis than the objective optical system.

### BRIEF DESCRIPTION OF THE DRAWINGS

FIGS. 1A and 1B are diagrams showing endoscope illumination systems according to an embodiment;

FIGS. 2A, 2B, and 2C are diagrams showing examples of an exit surface;

FIGS. 3A, 3B, and 3C show endoscope illumination systems and their light distributions;

FIGS. 4A, 4B, and 4C show endoscope illumination systems and their light distributions;

FIG. 5 is a diagram showing an endoscope illumination system according to the embodiment;

FIGS. 6A, 6B, and 6C show endoscope illumination systems and their light distributions;

FIGS. 7A and 7B are diagrams showing endoscope illumination systems according to the embodiment;

FIGS. 8A, 8B, and 8C show endoscope illumination systems and their light distributions;

FIGS. 9A, 9B, and 9C are diagrams illustrating parameters;

FIGS. 10A, 10B, and 10C show endoscope illumination systems and their light distributions;

FIGS. 11A, 11B, and 11C show endoscope illumination systems and their light distributions;

FIG. 12 is a diagram showing an endoscope illumination system according to the embodiment;

FIG. 13 is a diagram showing an endoscope illumination system according to the embodiment;

FIGS. 14A and 14B are diagrams illustrating an endoscope illumination system according to the embodiment;

FIGS. 15A and 15B are diagrams showing endoscope illumination systems according to the embodiment;

FIG. 16 is a diagram showing an endoscope illumination system according to the embodiment;

FIGS. 17A and 17B are diagrams showing endoscope illumination systems according to the embodiment;

FIGS. 18A and 18B illustrate an endoscope illumination system according to the embodiment and its light distribution;

FIGS. 19A and 19B illustrate an endoscope illumination system according to the embodiment and its light distribution;

FIG. 20 is a diagram showing an endoscope system;

FIG. 21 is a cross sectional view of the end portion of an insert part;

FIG. 22 is a diagram showing an endoscope illumination system according to the embodiment;

FIG. 23 is a diagram showing an endoscope illumination system according to the embodiment;

FIGS. 24A and 24B are diagrams showing an endoscope illumination system according to the embodiment.

### DETAILED DESCRIPTION OF THE INVENTION

In the following, an endoscope illumination system according to an embodiment and an endoscope according to an embodiment will be described in terms of the reason why they are configured as described and their operations. It should be understood that the present invention is not limited by the embodiments.

The endoscope illumination system according to the embodiment is an endoscope illumination system provided in an insert part. The endoscope illumination system has an exit surface from which illumination light is emitted, an entrance side optical surface on which the illumination light is incident, and an exit side optical surface from which the illumination light goes out. The entrance side optical surface has an inner light distribution surface and an outer light distribution surface. The outer light distribution surface is located farther from the center axis of the insert part than the inner light distribution surface. The inner light distribution surface has a first inner surface. The first inner surface is a curved surface that is convex to the exit surface. The outer light distribution surface is a flat surface or a curved surface that is concave toward the exit surface.

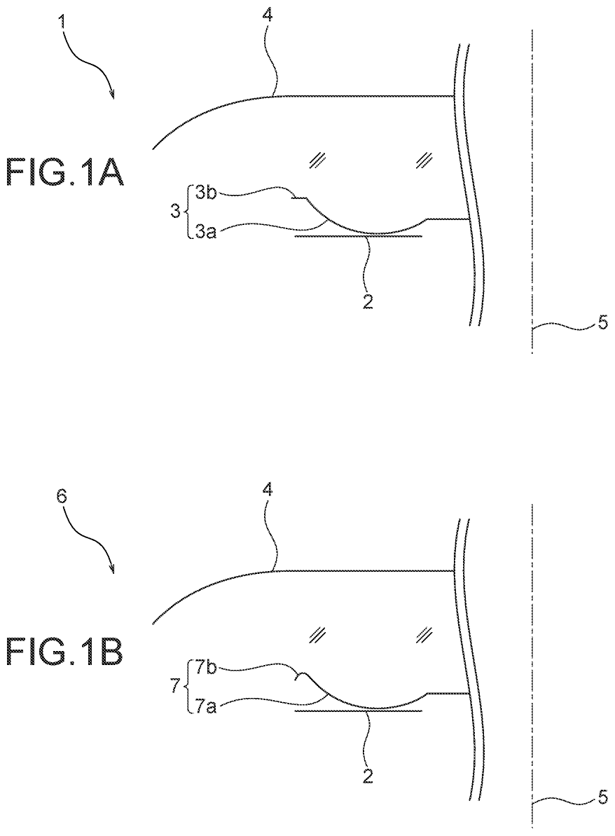

FIGS. 1A and 1B are diagrams showing endoscope illumination systems according to the embodiment. FIG. 1A is a diagram showing a first example of the endoscope illumination system according to the embodiment. FIG. 1B is a diagram showing a second example of the endoscope illumination system according to the embodiment.

The endoscope illumination system according to the embodiment is disposed in the end portion of the insert part of an endoscope. FIGS. 1A and 1B are cross sectional views of the end portion of the insert part. The detailed structure of the end portion of the insert part will be described later.

The endoscope illumination system 1 shown in FIG. 1A is the first exemplary endoscope illumination system. The endoscope illumination system 6 shown in FIG. 1B is the second exemplary endoscope illumination system. The endoscope illumination systems 1 and 6 have an exit surface 2. In the endoscope illumination systems 1 and 6, illumination light is emitted from the exit surface 2.

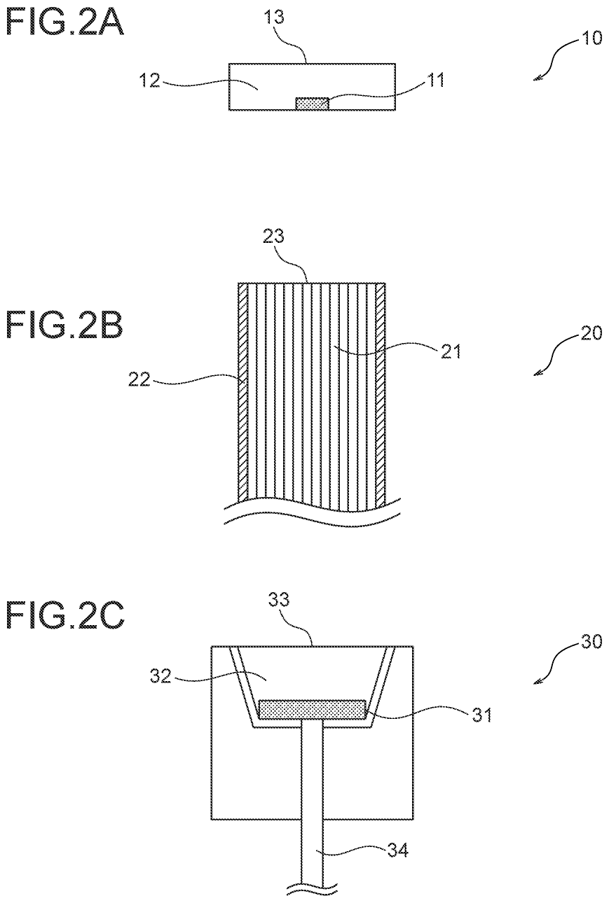

FIGS. 2A, 2B, and 2C show examples of the exit surface. FIG. 2A shows a first exemplary exit surface. FIG. 2B shows a second exemplary exit surface. FIG. 2C shows a third exemplary exit surface.

The first exemplary exit surface is an exit surface of alight emitting element. As illustrated in FIG. 2A, the light emitting element 10 has a light emitting part 11 and a sealing resin 12. Examples of the light emitting element 10 include an LED (Light Emitting Diode) and an LD (Laser Diode). The exit surface 13 is a surface of the sealing resin 12.

Light emitted from the light emitting part 11 travels through the sealing resin 12 to reach the exit surface 13. The light that reaches the exit surface 13 goes out from the exit surface 13.

The second exemplary exit surface is the end face of a light guide. As illustrated in FIG. 2B, the light guide 20 includes a fiber bundle 21 and a protection tube 22. The file bundle 21 is made up of a plurality of optical fibers. The exit surface 23 is the end face of the fiber bundle 21.

Light emitted from a light source (not shown) travels through the light guide 20 to reach the exit surface 23. The light that reaches the exit surface 23 goes out from the exit surface 23.

The third exemplary exit surface is the exit surface of an illumination unit. As illustrated in FIG. 2C, the illumination unit 30 includes a fluorescent element 31 and a sealing resin 32. The exit surface 33 is a surface of the sealing resin 32.

To the fluorescent element 31 is connected an optical fiber 34. Light emitted from a light source (not shown) travels through the optical fiber 34 to reach the fluorescent element 31. The fluorescent element 31 emits light emitted from the light source and fluorescent light. The wavelength of the fluorescent light is longer than the wavelength of the light emitted from the light source.

The light emitted from the fluorescent element 31 travels though the sealing resin 32 to reach the exit surface 33. The light that reaches the exit surface 33 goes out from the exit surface 33.

Referring back to FIGS. 1A and 1B, the endoscope illumination system 1 further includes an entrance side optical surface 3 and an exit side optical surface 4, as shown in FIG. 1A. The entrance side optical surface 3 in the endoscope illumination system 1 is opposed to the exit surface 2. Illumination light emitted from the exit surface 2 is incident on the entrance side optical surface 3.

The Entrance side optical surface 3 includes an inner light distribution surface 3a and an outer light distribution surface 3b. The outer light distribution surface 3b is located farther from a center axis 5 than the inner light distribution surface 3a. The center axis 5 is the center axis of the insert part.

The inner light distribution surface 3a includes a first inner surface. The first inner surface is a curved surface that is convex to the exit surface 2. In the case shown in FIG. 1A, the inner light distribution surface 3a is constituted only by a curved surface that is convex to the exit surface 2, that is, the inner light distribution surface 3a is constituted only by the first inner surface.

Referring to FIG. 1B, the endoscope illumination system 6 further has an entrance side optical surface 7 and an exit side optical surface 4. The entrance side optical surface 7 in the endoscope illumination system 6 is opposed to the exit surface 2. The illumination light emitted from the exit surface 2 is incident on the entrance side optical surface 7.

The entrance side optical surface 7 includes an inner light distribution surface 7a and an outer light distribution surface 7b. The outer light distribution surface 7b is located farther from the center axis 5 than the inner light distribution surface 7a.

The inner light distribution surface 7a includes a first inner surface. The first inner surface is a curved surface that is convex to the exit surface 2. In the case shown in FIG. 1B, the inner light distribution surface 7a is constituted only by a curved surface that is convex to the exit surface 2, that is, the inner light distribution surface 7a is constituted only by the first inner surface.

The first inner surface has a shape like, for example, a surface cut out from a toroidal surface. The toroidal surface is the surface of a body of revolution formed by revolving a circle on a plane about a straight line on the same plane that does not intersect with the circle as the axis of revolution.

The outer light distribution surface in the endoscope illumination system of this embodiment is a flat surface or a curved surface that is concave to the exit surface. In the case of the endoscope illumination system 1 shown in FIG. 1A, the outer light distribution surface 3b is a flat surface. In the case of the endoscope illumination system 6 shown in FIG. 1B, the outer light distribution surface 7b is a curved surface that is concave to the exit surface 2.

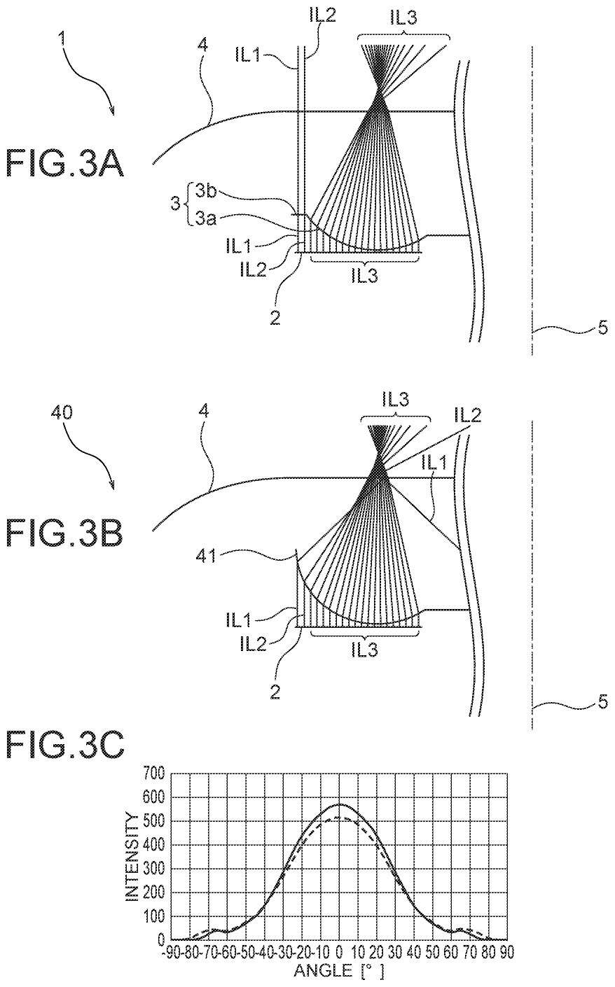

FIGS. 3A, 3B, and 3C shows endoscope illumination systems and their light distributions. FIG. 3A is a diagram showing the first exemplary endoscope illumination system. FIG. 3B is a diagram showing a conventional endoscope illumination system. FIG. 3C is a graph showing light distributions of illumination light. The elements same as those in FIG. 1A are denoted by the same reference numerals and will not be described further.

Illumination light is emitted from the exit surface in various directions. FIGS. 3A and 3B show only light that goes out from the exit surface parallel to the center axis.

The endoscope illumination system 1 will be described with reference to FIG. 3A. As described above, the endoscope illumination system 1 is the first example of the endoscope illumination system according to the embodiment.

Illumination light emitted from the exit surface 2 includes illumination light IL1, illumination light IL2, and illumination light IL3. The illumination light IL1, the illumination light IL2, and the illumination light IL3 are incident on the entrance side optical surface 3. The entrance side optical surface 3 includes the inner light distribution surface 3a and the outer light distribution surface 3b.

The illumination light IL3 is incident on the inner light distribution surface 3a. The inner light distribution surface 3a is a curved surface. In consequence, the illumination light IL3 is refracted on the inner light distribution surface 3a to converge.

The illumination light IL1 and the illumination light IL2 are incident on the outer light distribution surface 3b. The outer light distribution surface 3b is a flat surface. In consequence, the illumination light IL1 and the illumination light IL2 are not refracted on the outer light distribution surface 3b and travel parallel to the center axis 5.

The space between the entrance side optical surface 3 and the exit side optical surface 4 is filled with, for example, a transparent medium having a refractive index larger than 1. The illumination light IL1, the illumination light IL2, and the illumination light IL3 travel through the transparent medium to reach the exit side optical surface 4.

The illumination light IL1, the illumination light IL2, and the illumination light IL3 are incident on a flat portion of the exit side optical surface 4. The illumination light IL1 and the illumination light IL2 are not refracted on the exit side optical surface 4 and travel parallel to the center axis 5. The illumination light IL3 converges and then diverges.

Another endoscope illumination system 40 will be described with reference to FIG. 3B. The endoscope illumination system 40 shown in FIG. 3B is a conventional endoscope illumination system. This endoscope illumination system 40 has an exit surface 2, an entrance side optical surface 41, and an exit side optical surface 4.

Illumination light emitted from the exit surface 2 includes illumination light IL1, illumination light IL2, and illumination light IL3. The entrance side optical surface 41 is opposed to the exit surface 2. The illumination light IL1, the illumination light IL2, and the illumination light IL3 are incident on the entrance side optical surface 41.

The entrance side optical surface 41 is constituted only by a curved surface that is convex to the exit surface 2. In consequence, the illumination light IL1 and the illumination light IL2 are refracted on the curved surface and travel in directions crossing the center axis 5. The illumination light IL3 is refracted on the curved surface to converge.

The illumination light IL1 and the illumination light IL2 are farther from the center axis 5 than the illumination light IL3. In consequence, the angle of incidence of the illumination light IL1 and the illumination light IL2 on the entrance side optical surface 41 is larger than that of the illumination light IL3. Therefore, illumination light IL1 and the illumination light IL2 are refracted more greatly than the illumination light IL3.

The space between the entrance side optical surface 41 and the exit side optical surface 4 is filled with, for example, a transparent medium having a refractive index larger than 1. The illumination light IL1, the illumination light IL2, and the illumination light IL3 travel through the transparent medium to reach the exit side optical surface 4.

The illumination light IL1, the illumination light IL2, and the illumination light IL3 are incident on a flat portion of the exit side optical surface 4. The illumination light IL1 is totally reflected by the exit side optical surface 4. The illumination light IL2 is further refracted on the exit side optical surface 4 and travels in a direction crossing the center axis 5. The illumination light IL3 converges and then diverges.

In the case of the endoscope illumination system 40, the illumination light IL1 is refracted on the entrance side optical surface 41 and then reflected by the exit side optical surface 4. Therefore, the illumination light IL1 does not go out from the exit side optical surface 4. The illumination light IL2 is refracted on both the entrance side optical surface 41 and the exit side optical surface 4 and travels in a direction crossing the center axis 5. Thus, the illumination light IL2 goes out from the exit side optical surface 4. However, the illumination light IL 2 is refracted so largely on the entrance side optical surface 41 that the illumination light IL 2 is cast outside the observation area of the endoscope. This leads to a decrease in the illumination efficiency.

In contrast, in the case of the endoscope illumination system 1 shown in FIG. 3A, the illumination light IL1 and the illumination light IL2 are not refracted on the entrance side optical surface 3 nor the exit side optical surface 4 but travel parallel to the center axis 5. Therefore, the illumination light IL1 and the illumination light IL 2 go out from the exit side optical surface 4. Moreover, the illumination light IL1 and the illumination light IL 2 are not cast outside the observation area. In consequence, it is possible to prevent a decrease in the illumination efficiency.

In FIG. 3C, the solid curve represents the distribution of illumination light with the endoscope illumination system 1, and the broken curve represents the distribution of illumination light with the endoscope illumination system 40. FIG. 3C shows the light distributions in the case where the endoscope illumination systems are arranged symmetrically with respect to the center axis 5. The horizontal axis represents the angle, and the vertical axis represents the intensity.

In the case of the endoscope illumination system 1, the angle at which the intensity becomes zero is smaller than 80°. In contrast, in the case of the endoscope illumination system 40, the angle at which the intensity becomes zero is larger than 80°. Since it is considered that the range of the angle represents the extent of the illumination area, FIG. 3C indicates that the illumination area of the endoscope illumination system 1 is smaller than the illumination area of the endoscope illumination system 40.

The smaller illumination area means a smaller quantity of illumination light that is cast outside the observation area. Therefore, the endoscope illumination system 1 can illuminate the observation area more efficiently than the endoscope illumination system 40.

As described above, the outer light distribution surface 3b is located farther from the center axis 5 than the inner light distribution surface 3a. In the case where the center of the observation area is located on the center axis 5, the illumination light IL1 and the illumination light IL2 are incident on the peripheral portion of the observation area (i.e. the peripheral area inside the observation area). Thus, the peripheral portion of the observation area can be illuminated brightly.

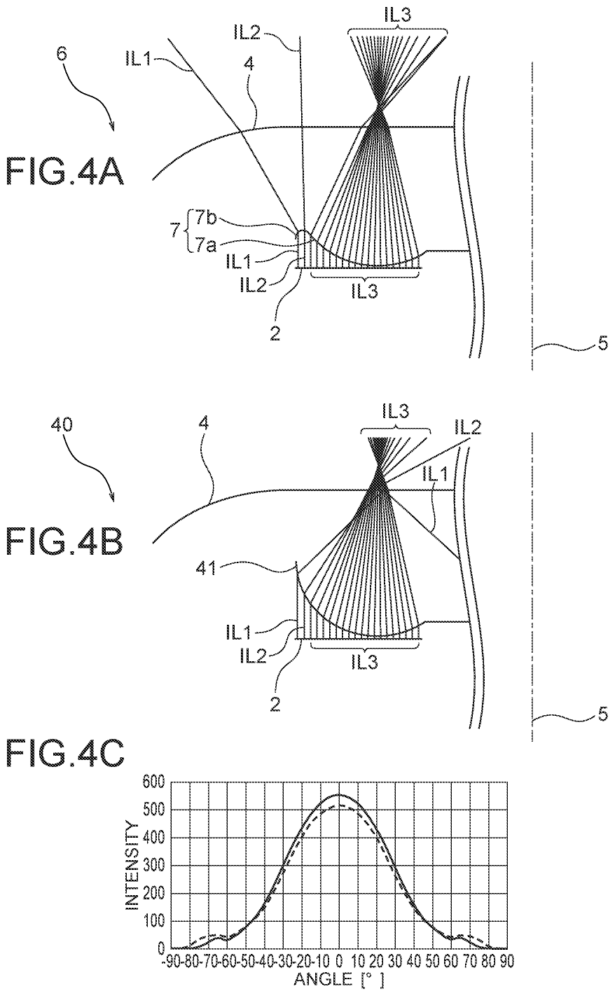

FIGS. 4A, 4B, and 4C show endoscope illumination systems and their light distributions. FIG. 4A is a diagram showing the second exemplary endoscope illumination system. FIG. 4B is a diagram showing the conventional endoscope illumination system. FIG. 4C is a graph showing light distributions of illumination light. The elements same as those in FIG. 1B are denoted by the same reference numerals and will not be described further. FIG. 4B is the same as FIG. 3B.

While illumination light goes out from the exit surface in various directions, FIGS. 4A and 4B show only illumination light that goes out from the exit surface parallel to the center axis.

The endoscope illumination system 6 will be described with reference to FIG. 4A. As described above, the endoscope illumination system 6 is the second example of the endoscope illumination system according to the embodiment.

Illumination light emitted from the exit surface 2 includes illumination light IL1, illumination light IL2, and illumination light IL3. The illumination light IL1, the illumination light IL2, and the illumination light IL3 are incident on the entrance side optical surface 7.

The entrance side optical surface 7 includes the inner light distribution surface 7a and the outer light distribution surface 7b. The illumination light IL3 is incident on the inner light distribution surface 7a. The inner light distribution surface 7a is a curved surface. In consequence, the illumination light IL3 is refracted on the inner light distribution surface 7a to converge.

The illumination light IL1 and the illumination light IL2 are incident on the outer light distribution surface 7b. The outer light distribution surface 7b is a curved surface that is concave to the exit surface 2. In consequence, the illumination light IL1 and the illumination light IL2 are refracted on the outer light distribution surface 7b. The illumination light IL1 travels away from the center axis 5, and the illumination light IL2 travels substantially parallel to the center axis 5.

The space between the entrance side optical surface 7 and the exit side optical surface 4 is filled with, for example, a transparent medium having a refractive index larger than 1. The illumination light IL1, the illumination light IL2, and the illumination light IL3 travel through the transparent medium to reach the exit side optical surface 4.

The illumination light IL1, the illumination light IL2, and the illumination light IL3 are incident on a flat portion of the exit side optical surface 4. The illumination light IL1, the illumination light IL2, and the illumination light IL3 are refracted on the exit side optical surface 4. The illumination light IL1 travels away from the center axis 5. The illumination light IL2 travels substantially parallel to the center axis 5. The illumination light IL3 converges and then diverges.

As described above, in the case of the endoscope illumination system 40, the illumination light IL1 does not go out from the exit side optical surface 4. The illumination light IL2 goes out from the exit side optical surface 4, but it is cast outside the observation area. This leads to a decrease in the illumination efficiency.

In contrast, in the case of the endoscope illumination system 40, the illumination light IL1 and the illumination light IL2 are refracted on both the entrance side optical surface 7 and the exit side optical surface 4. However, the illumination light IL1 is not refracted so greatly as the illumination light IL1 in the endoscope illumination system 40. The illumination light IL2 travels substantially parallel to the center axis 5. In consequence, the illumination light IL1 and the illumination light IL2 go out from the exit side optical surface 4. Moreover, the illumination light IL1 and the illumination light IL2 are not cast outside the observation area. In consequence, it is possible to prevent a decrease in the illumination efficiency.

In FIG. 4C, the solid curve represents the distribution of illumination light with the endoscope illumination system 6, and the broken curve represents the distribution of illumination light with the endoscope illumination system 40. FIG. 4C shows the light distributions in the case where the endoscope illumination systems are arranged symmetrically with respect to the center axis 5. The horizontal axis represents the angle, and the vertical axis represents the intensity.

In the case of the endoscope illumination system 6, the angle at which the intensity becomes zero is smaller than 80°. In contrast, in the case of the endoscope illumination system 40, the angle at which the intensity becomes zero is larger than 80°. FIG. 4C indicates that the illumination area of the endoscope illumination system 6 is smaller than the illumination area of the endoscope illumination system 40.

The smaller illumination area means a smaller quantity of illumination light that is cast outside the observation area. Therefore, the endoscope illumination system 6 can illuminate the observation area more efficiently than the endoscope illumination system 40.

As described above, the outer light distribution surface 7b is located farther from the center axis 5 than the inner light distribution surface 7a. In the case where the center of the observation area is located on the center axis 5, the illumination light IL1 and the illumination light IL2 are incident on the peripheral portion of the observation area. Thus, the peripheral portion of the observation area can be illuminated brightly.

It is preferred that the inner light distribution surface in the endoscope illumination system according to the embodiment include a first inner surface and a second inner surface, the second inner surface be a flat surface, and the second inner surface be located closer to the center axis than the first inner surface.

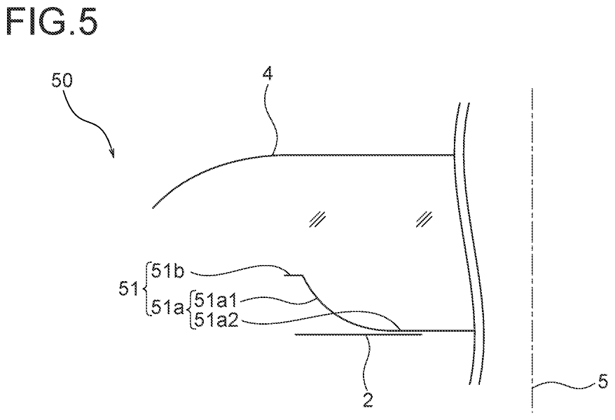

FIG. 5 is a diagram showing another endoscope illumination system according to the embodiment. FIG. 5 shows a third example of the endoscope illumination system according to the embodiment. The elements same as those in FIG. 1A are denoted by the same reference numerals and will not be described further.

The endoscope illumination system 50 is the third exemplary endoscope illumination system. The endoscope illumination system 50 includes an exit surface 2, an entrance side optical surface 51, and an exit side optical surface 4. In the endoscope illumination system 50, illumination light is emitted from the exit surface 2.

Illumination light emitted from the exit surface 2 is incident on the entrance side optical surface 51. The entrance side optical surface 51 includes an inner light distribution surface 51a and an outer light distribution surface 51b. The outer light distribution surface 51b is located farther from the center axis 5 than the inner light distribution surface 51a.

The inner light distribution surface 51a includes a first inner surface 51a1 and a second inner surface 51a2. The first inner surface 51a1 is a curved surface that is convex to the exit surface 2. The second inner surface 51a2 is a flat surface. The second inner surface 51a2 is located closer to the center axis 5 than the first inner surface 51a1.

The outer light distribution surface in the endoscope illumination system according to the embodiment is a flat surface or a curved surface that is concave to the exit surface. In the case of this endoscope illumination system 50, the outer light distribution surface 51b is a flat surface.

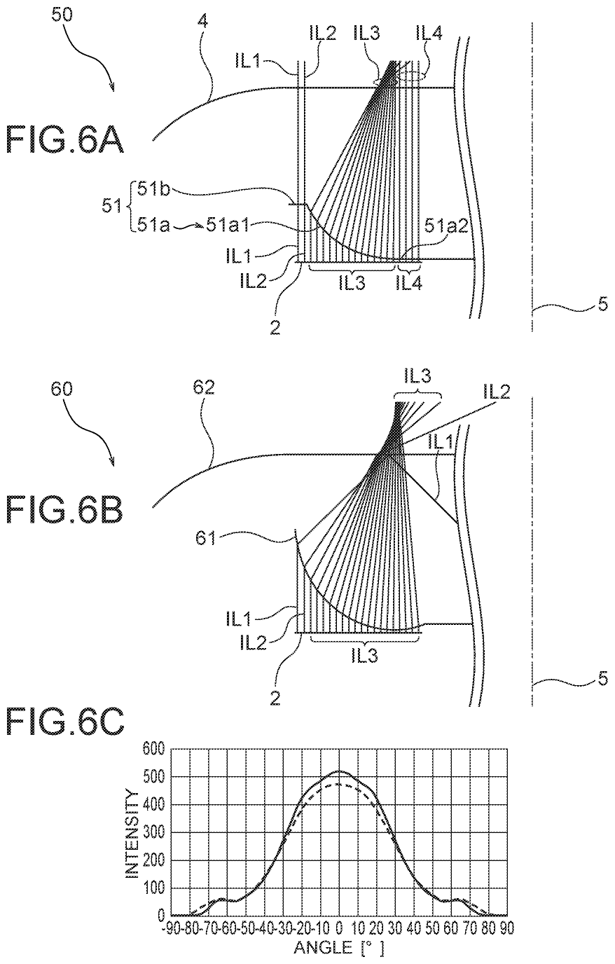

FIG. 6 shows endoscope illumination systems and their light distributions. FIG. 6A is a diagram showing the third exemplary endoscope illumination system. FIG. 6B is a diagram showing a conventional endoscope illumination system. FIG. 6C is a graph showing light distributions of illumination light. The elements same as those in FIG. 5 are denoted by the same reference numerals and will not be described further.

While illumination light is emitted from the exit surface in various directions, FIGS. 6A and 6B show only illumination light that goes out from the exit surface parallel to the center axis.

The endoscope illumination system 50 will be described with reference to FIG. 6A. As described above, the endoscope illumination system 50 is the third example of the endoscope illumination system according to the embodiment.

Illumination light emitted from the exit surface 2 includes illumination light IL1, illumination light IL2, illumination light IL3, and illumination light IL4. The illumination light IL1, the illumination light IL2, the illumination light IL3, and the illumination light IL4 are incident on the entrance side optical surface 51. The entrance side optical surface 51 includes an inner light distribution surface 51a and an outer light distribution surface 51b.

The illumination light IL3 and the illumination light IL4 are incident on the inner light distribution surface 51a. The inner light distribution surface 51a includes a first inner surface 51a1 and a second inner surface 51a2.

The illumination light IL3 is incident on the first inner surface 51a1. The first inner surface 51a1 is a curved surface. In consequence, the illumination light IL3 is refracted on the first inner surface 51a1 to converge.

The illumination light IL4 is incident on the second inner surface 51a2. The second inner surface 51a2 is a flat surface. In consequence, the illumination light IL4 is not refracted on the second inner surface 42b and travels parallel to the center axis 5.

The illumination light IL1 and the illumination light IL2 are incident on the outer light distribution surface 51b. The outer light distribution surface 51b is a flat surface. In consequence, the illumination light IL1 and the illumination light IL2 are not refracted on the outer light distribution surface 51b and travel parallel to the center axis 5.

The space between the entrance side optical surface 51 and the exit side optical surface 4 is filled with, for example, a transparent medium having a refractive index larger than 1. The illumination light IL1, the illumination light IL2, the illumination light IL3, and the illumination light IL4 travel through the transparent medium to reach the exit side optical surface 4.

The illumination light IL1, the illumination light IL2, the illumination light IL3, and the illumination light IL4 are incident on a flat portion of the exit side optical surface 4. The illumination light IL1, the illumination light IL2, and the illumination light IL4 are not refracted on the exit side optical surface 4 and travel parallel to the center axis 5. The illumination light IL3 converges and then diverges.

Another endoscope illumination system 60 will be described with reference to FIG. 6B. The endoscope illumination system 60 shown in FIG. 6B is a conventional endoscope illumination system. This endoscope illumination system 60 has an exit surface 2, an entrance side optical surface 61, and an exit side optical surface 62.

Illumination light emitted from the exit surface 2 includes illumination light IL1, illumination light IL2, and illumination light IL3. The entrance side optical surface 61 is opposed to the exit surface 2. The illumination light IL1, the illumination light IL2, and the illumination light IL3 are incident on the entrance side optical surface 61.

The entrance side optical surface 61 is constituted only by a curved surface that is convex to the exit surface 2. In consequence, the illumination light IL1 and the illumination light IL2 are refracted on the curved surface and travel in directions crossing the center axis 5. The illumination light IL3 is refracted on the curved surface to converge.

The illumination light IL1 and the illumination light IL2 are farther from the center axis 5 than the illumination light IL3. In consequence, the angle of incidence of the illumination light IL1 and the illumination light IL2 on the entrance side optical surface 61 is larger than that of the illumination light IL3. Therefore, illumination light IL1 and the illumination light IL2 are refracted more greatly than the illumination light IL3.

The space between the entrance side optical surface 61 and the exit side optical surface 62 is filled with, for example, a transparent medium having a refractive index larger than 1. The illumination light IL1, the illumination light IL2, and the illumination light IL3 travel through the transparent medium to reach the exit side optical surface 62.

The illumination light IL1, the illumination light IL2, and the illumination light IL3 are incident on a flat portion of the exit side optical surface 62. The illumination light IL1 is totally reflected by the exit side optical surface 62. The illumination light IL2 is further refracted on the exit side optical surface 62 and travels in a direction crossing the center axis 5. The illumination light IL3 converges and then diverges.

In the case of the endoscope illumination system 60, the illumination light IL1 does not go out from the exit side optical surface 60, like in the case of the endoscope illumination system 40. The illumination light IL2 goes out from the exit side optical surface 62. However, the illumination light IL2 is refracted so largely on the entrance side optical surface 61 that the illumination light IL2 is cast outside the observation area of the endoscope. This leads to a decrease in the illumination efficiency.

In contrast, in the case of the endoscope illumination system 50, the illumination light IL1 and the illumination light IL2 are not refracted on the entrance side optical surface 51 nor the exit side optical surface 4 but travel parallel to the center axis 5. In consequence, the illumination light IL1 and the illumination light IL 2 go out from the exit side optical surface 4. Moreover, the illumination light IL1 and the illumination light IL 2 are not cast outside the observation area. In consequence, it is possible to prevent a decrease in the illumination efficiency.

In FIG. 6C, the solid curve represents the distribution of illumination light with the endoscope illumination system 1, and the broken curve represents the distribution of illumination light with the endoscope illumination system 60. FIG. 6C shows the light distributions in the case where the endoscope illumination systems are arranged symmetrically with respect to the center axis 5. The horizontal axis represents the angle, and the vertical axis represents the intensity.

In the case of the endoscope illumination system 1, the angle at which the intensity becomes zero is smaller than 80°. In contrast, in the case of the endoscope illumination system 60, the angle at which the intensity becomes zero is approximately 80°. Since it is considered that the range of the angle represents the extent of the illumination area, FIG. 6C indicates that the illumination area of the endoscope illumination system 50 is smaller than the illumination area of the endoscope illumination system 60.

The smaller illumination area means a smaller quantity of illumination light that is cast outside the observation area. Therefore, the endoscope illumination system 50 can illuminate the observation area more efficiently than the endoscope illumination system 60.

As described above, the outer light distribution surface 51b is located farther from the center axis 5 than the inner light distribution surface 51a. In the case where the center of the observation area is located on the center axis 5, the illumination light IL1 and the illumination light IL2 are incident on the peripheral portion of the observation area. Thus, the peripheral portion of the observation area can be illuminated brightly.

The illumination light IL4 is also incident perpendicularly on both the outer light distribution surface 51b and the exit side optical surface 4. In consequence, the illumination light is not refracted on the outer light distribution surface 51b nor the exit side optical surface 4 but travels parallel to the center axis 5. The illumination light IL4 travels near the center axis 5. Therefore, the illumination light IL4 is incident on the central portion of the observation area. In consequence, it is possible to illuminate the central portion of the observation area brightly without decreasing the illumination efficiency.

It is preferred in the endoscope illumination system according to the embodiment that the exit side optical surface include a first exit side surface and a second exit side surface, the first exit side surface be a flat surface, the second exit side surface be a curved surface, the second exit side surface be located farther from the center axis than the first exit side surface, and the straight line that is parallel to the center axis and passes the boundary of the first exit side surface and the second exit side surface intersects with the exit surface.

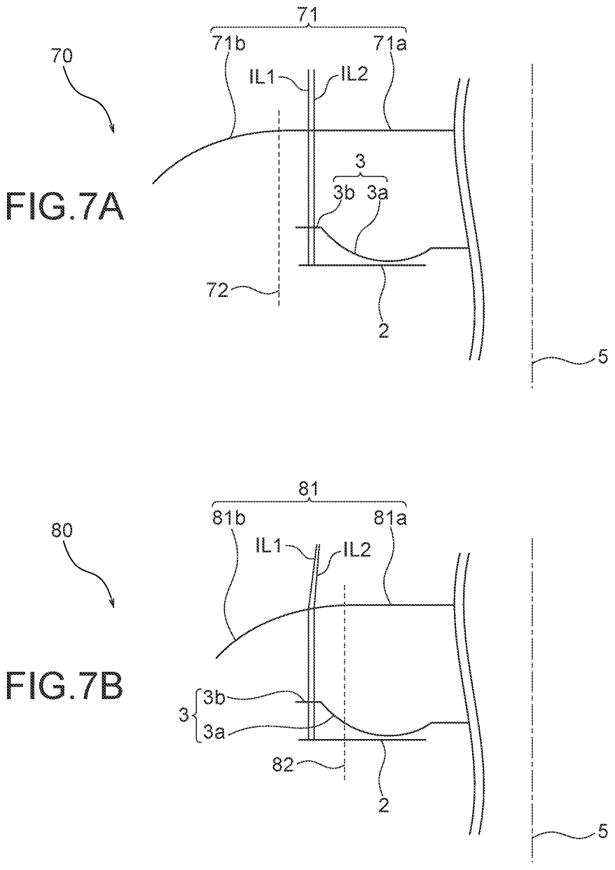

FIG. 7 shows endoscope illumination systems according to the embodiment. FIG. 7A is a diagram showing the first exemplary endoscope illumination system. FIG. 7B is a diagram showing a fourth exemplary endoscope illumination system. The elements same as those in FIG. 1A are denoted by the same reference numerals and will not be described further.

FIGS. 7A and 7B show only a portion of the illumination light emitted from the exit surface. While illumination light goes out from the exit surface in various directions, FIGS. 7A and 7B show only illumination light that goes out from the exit surface parallel to the center axis.

The endoscope illumination system 70 will be described with reference to FIG. 7A. The endoscope illumination system 70 is the first exemplary endoscope illumination system according to the embodiment.

The exit side optical surface 71 includes a first exit side surface 71a and a second exit side surface 71b. The first exit side surface 71a is a flat surface. The second exit side surface 71b is a curved surface.

The second exit side surface 71b is located farther from the center axis 5 than the first exit side surface 71a. The line denoted by the reference numeral “72” is a straight line that is parallel to the center axis 5 and passes the boundary of the first exit side surface 71a and the second exit side surface 71b.

In the case of this endoscope illumination system. 70, the straight line 72 does not intersect with the exit surface 2. In this case, the illumination light IL1 and the illumination light IL2 are incident on the first exit side surface 71a. The first exit side surface 71a is a flat surface. In consequence, the illumination light IL1 and the illumination light IL2 are not refracted on the exit side optical surface 71 but travel parallel to the center axis 5.

Another endoscope illumination system 80 will be described with reference to FIG. 7B. The endoscope illumination system 80 is the fourth example of the endoscope illumination system according to the embodiment.

The exit side optical surface 81 includes a first exit side surface 81a and a second exit side surface 81b. The first exit side surface 81a is a flat surface. The second exit side surface 81b is a curved surface.

The second exit side surface 81b is located farther from the center axis 5 than the first exit side surface 81a. The line denoted by the reference numeral “82” is a straight line that is parallel to the center axis 5 and passes the boundary of the first exit side surface 81a and the second exit side surface 81b.

In the case of this endoscope illumination system. 80, the straight line 82 intersects with the exit surface 2. In this case, the illumination light IL1 and the illumination light IL2 are incident on the second exit side surface 81b. The second exit side surface 81b is a curved surface. In consequence, the illumination light IL1 and the illumination light IL2 are refracted on the exit side optical surface 81 and travel in directions crossing the center axis 5.

Refraction of the illumination light IL1 and the illumination light IL2 occurs only on the second exit side surface 81b. In this case, the illumination light IL1 and the illumination light IL2 are not refracted so greatly as in conventional endoscope illumination systems. Hence, the illumination light IL1 and the illumination light IL2 are not cast outside the observation area. In consequence, it is possible to prevent a decrease in the illumination efficiency.

As described above, the second exit side surface 81b is located farther from the center axis 5 than the first exit side surface 81a. In the case where the center of the observation area is located on the center axis 5, the illumination light IL1 and the illumination light IL2 are incident on the central portion of the observation area. In consequence, it is possible to illuminate the central portion of the observation area brightly.

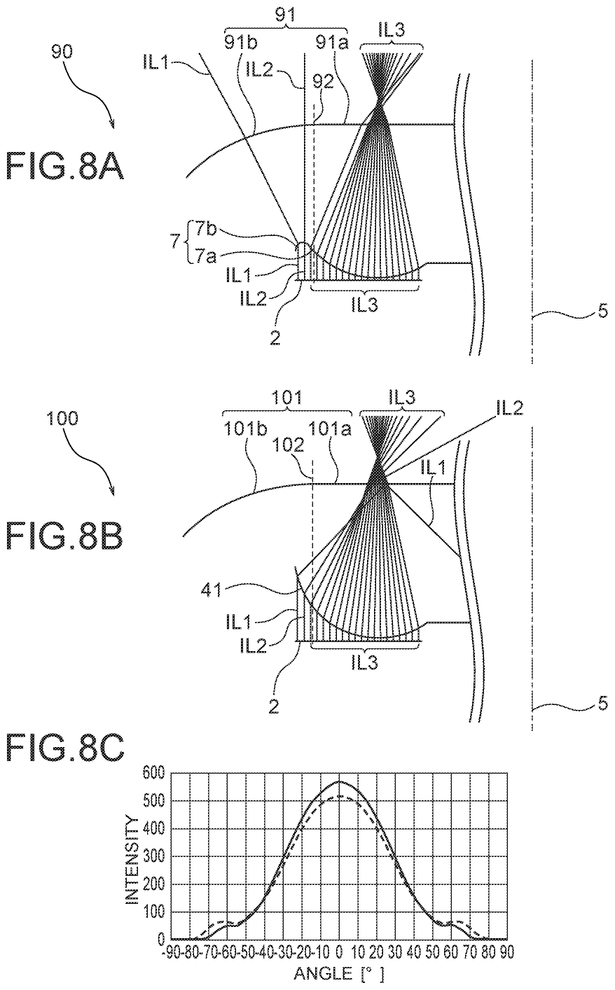

FIG. 8 shows endoscope illumination systems and their light distributions. FIG. 8A is a diagram showing a fifth exemplary endoscope illumination system. FIG. 8B is a diagram showing a conventional endoscope illumination system. FIG. 8C is a graph showing light distributions of illumination light. The elements same as those in FIGS. 4A and 4B are denoted by the same reference numerals and will not be described further.

While illumination light goes out from the exit surface in various directions, FIGS. 8A and 8B show only illumination light that goes out from the exit surface parallel to the center axis.

The endoscope illumination system 80 will be described with reference to FIG. 8A. The endoscope illumination system 80 is the fifth exemplary endoscope illumination system according to the embodiment.

Illumination light emitted from the exit surface 2 includes illumination light IL1, illumination light IL2, and illumination light IL3. The illumination light IL1, the illumination light IL2, and the illumination light IL3 are incident on the entrance side optical surface 7.

The entrance side optical surface 7 includes an inner light distribution surface 7a and an outer light distribution surface 7b. The illumination light IL3 is incident on the inner light distribution surface 7a. The illumination light IL1 and the illumination light IL2 are incident on the outer light distribution surface 7b.

The illumination light IL3 is refracted on the inner light distribution surface 7a to converge. The illumination light IL1 and the illumination light IL2 are refracted on the outer light distribution surface 7b. The illumination light IL1 travels away from the center axis 5, and the illumination light IL2 travels substantially parallel to the center axis.

The space between the entrance side optical surface 7 and the exit side optical surface 91 is filled with, for example, a transparent medium having a refractive index larger than 1. The illumination light IL1, the illumination light IL2, and the illumination light IL3 travel through the transparent medium to reach the exit side optical surface 91.

The exit side optical surface 91 includes a first exit side surface 91a and a second exit side surface 91b. The first exit side surface 91a is a flat surface and the second exit side surface 91b is a curved surface. The second exit side surface 91b is located farther from the center axis 5 than the first exit side surface 91a.

In the case of this endoscope illumination system. 90, the line denoted by the reference numeral “92” intersects with the exit surface 2. In this case, the second exit side surface 91b is located closer to the center axis 5 than that in the case where the straight line 92 does not intersect with the exit surface 2. In consequence, the illumination light IL1 and the illumination light IL2 are incident on the second exit side surface 91b, and the illumination light IL3 is incident on the first exit side surface 91a.

The illumination light IL1 is refracted on the second exit side surface 91b to travel away from the center axis 5. The illumination light IL2 is refracted on the second exit side surface 91b to travel substantially parallel to the center axis 5. The illumination light IL3 converges and then diverges.

If the second exit side surface 91b is a spherical surface, the center of curvature is close to the outer light distribution surface 7b. If the center of curvature is close to the outer light distribution surface 7b, the angle of incidence of the illumination light IL1 on the second exit side surface 91b is small. Then, the refraction of the illumination light IL1 on the second exit side surface 91b is small, and hence the illumination light IL1 is not cast outside the observation area. In consequence, it is possible to prevent a decrease in the illumination efficiency.

Another endoscope illumination system 100 will be described with reference to FIG. 8B. The endoscope illumination system 100 shown in FIG. 8B is a conventional endoscope illumination system. This endoscope illumination system has an exit surface 2, an entrance side optical surface 41, and an exit side optical surface 101.

Illumination light emitted from the exit surface 2 includes illumination light IL1, illumination light IL2, and illumination light IL3. The entrance side optical surface 41 is opposed to the exit surface 2. The illumination light IL1, the illumination light IL2, and the illumination light IL3 are incident on the entrance side optical surface 41.

The space between the entrance side optical surface 41 and the exit side optical surface 101 is filled with, for example, a transparent medium having a refractive index larger than 1. The illumination light IL1, the illumination light IL2, and the illumination light IL3 travel through the transparent medium to reach the exit side optical surface 101.

The exit side optical surface 101 includes a first exit side surface 101a and a second exit side surface 101b. The first exit side surface 101a is a flat surface, and the second exit side surface 101b is a curved surface. The second exit side surface 101b is located farther from the center axis 5 than the first exit side surface 101a.

In the case of this endoscope illumination system 100, the line denoted by the reference numeral “102” intersects with the exit surface 2. In this case, the second exit side surface 101b is located closer to the center axis 5 than that in the case where the straight line 102 does not intersect with the exit surface 2. However, the illumination light IL1, the illumination light IL2, and the illumination light IL3 are not incident on the second exit side surface 101b.

The illumination light IL1, the illumination light IL2, and the illumination light IL3 are incident on the first exit surface 101a. In other words, the illumination light IL1, the illumination light IL2, and the illumination light IL3 are incident on a flat surface. Then, the illumination light IL1 is totally reflected by the first exit side surface 101a. The illumination light IL2 is further refracted on the first exit side surface 101a and travels in a direction crossing the center axis 5. The illumination light IL3 converges and then diverges.

In the case of the endoscope illumination system. 100, the illumination light IL1 is refracted on the entrance side optical surface 41 and then reflected by the exit side optical surface 101. Thus, the illumination light IL1 does not go out from the exit side optical surface 101. The illumination light IL2 is refracted on both the entrance side optical surface 41 and the exit side optical surface 101 and travels in a direction crossing the center axis 5. Thus, the illumination light IL2 goes out from the exit side optical surface 91. However, the illumination light IL 2 is refracted so largely on the entrance side optical surface 41 that the illumination light IL 2 is cast outside the observation area of the endoscope. This leads to a decrease in the illumination efficiency.

In contrast, in the case of the endoscope illumination system 90, the illumination light IL1 and the illumination light IL2 are refracted on both the entrance side optical surface 7 and the exit side optical surface 91. However, the illumination light IL1 is not refracted so greatly as that in the endoscope illumination system 100. The illumination light IL2 travels substantially parallel to the center axis 5. In consequence, the illumination light IL1 and the illumination light IL2 go out from the exit side optical surface 91. Moreover, the illumination light IL1 and the illumination light IL2 are not cast outside the observation area. In consequence, it is possible to prevent a decrease in the illumination efficiency.

In FIG. 8C, the solid curve represents the distribution of illumination light with the endoscope illumination system 90, and the broken curve represents the distribution of illumination light with the endoscope illumination system. 100. FIG. 8C shows the light distributions in the case where the endoscope illumination systems are arranged symmetrically with respect to the center axis 5. The horizontal axis represents the angle, and the vertical axis represents the intensity.

In the case of the endoscope illumination system 90, the angle at which the intensity becomes zero is approximately 70°. In contrast, in the case of the endoscope illumination system 100, the angle at which the intensity becomes zero is larger than 70°. FIG. 8C indicates that the illumination area of the endoscope illumination system 90 is smaller than the illumination area of the endoscope illumination system 100.

The smaller illumination area means a smaller quantity of illumination light that is cast outside the observation area. Therefore, the endoscope illumination system 90 can illuminate the observation area more efficiently than the endoscope illumination system 100.

As described above, the outer light distribution surface 7b is located farther from the center axis 5 than the inner light distribution surface 7a. In the case where the center of the observation area is located on the center axis 5, the illumination light IL1 and the illumination light IL2 are incident on the peripheral portion of the observation area. Thus, the peripheral portion of the observation area can be illuminated brightly.

It is preferred that the endoscope illumination system according to the embodiment satisfy the following conditional expression (1):

8≤d1/d2≤32 (1)

where d1 is the width of the inner light distribution surface, and d2 is the width of the outer light distribution surface.

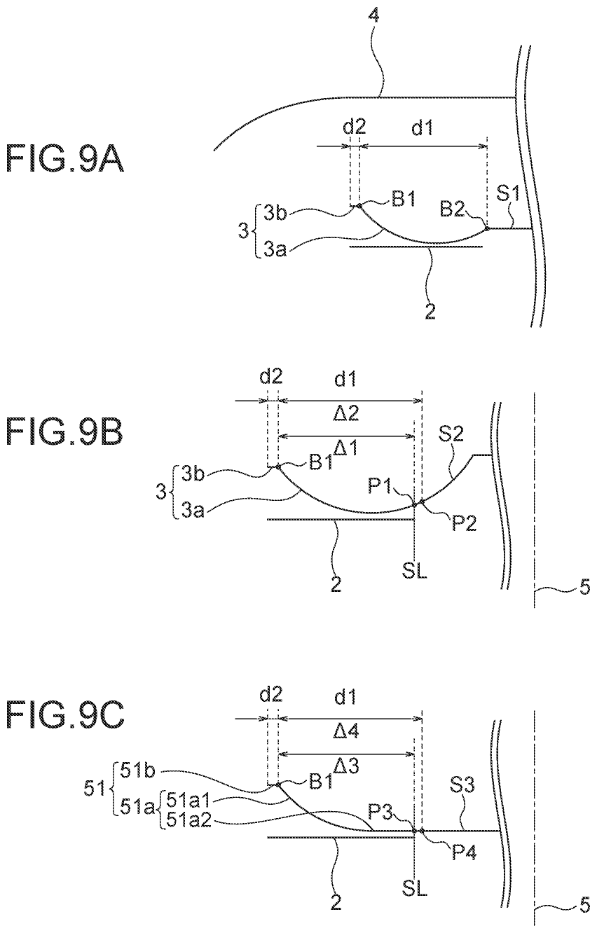

FIGS. 9A, 9B, and 9C are diagrams illustrating various parameters. FIGS. 9A, 9B, and 9C are cross sectional views taken on a plane containing the center axis of the insert part. FIG. 9A is a diagram showing a first example of the inner light distribution surface. FIG. 9B is a diagram showing a second example of the inner light distribution surface. FIG. 9C is a diagram showing a third example of the inner light distribution surface. The elements same as those in FIGS. 1A and 5 are denoted by the same reference numerals and will not be described further.

What is denoted by d1 is the width of the inner light distribution surface. What is denoted by d2 is the width of the outer light distribution surface. The widths d1 and d2 are lengths on a cross section containing the center axis of the insert part.

The first example of the inner light distribution surface will be described here with reference to FIG. 9A. In the case of the first example of the inner light distribution surface, the inner light distribution surface 3a abuts on the outer light distribution surface 3b and a surface S1. The inner light distribution surface 3a is a curved surface. The outer light distribution surface 3b and the surface S1 are flat surfaces. In this case, the boundary B1 of the inner light distribution surface 3a and the outer light distribution surface 3b and the boundary B2 of the inner light distribution surface 3a and the surface S1 are distinct. Therefore, the width d1 of the first exemplary light distribution surface can be determined based on the boundary B1 and the boundary B2.

Next, the second example of the inner light distribution surface will be described with reference to FIG. 9B. In the case of the second example of the inner light distribution surface, the inner light distribution surface 3a abuts on the outer light distribution surface 3B and a surface S2.

The inner light distribution surface 3a is a curved surface. The outer light distribution surface 3b is a flat surface, and therefore the boundary B1 of the inner light distribution surface 3a and the outer light distribution surface 3b is distinct. The surface S2 is a curved surface like the inner light distribution surface 3a, and therefore the boundary of the inner light distribution surface 3a and the flat surface S2 is not distinct. For this reason, the width d1 cannot be determined based on the boundaries in the case of the second example of the inner light distribution surface. In the case of the second example of the inner light distribution surface, the width d1 is determined based on the boundary B1 and the position P1 or on the boundary B1 and the position P2.

In the case where the width d1 is determined based on the boundary B1 and the position P1, the width d1 is expressed as the distance Δ1 between the boundary B1 and the position P1. The position P1 is the point of intersection of the inner light distribution surface 3a and the straight line SL. The straight line SL is a line that is parallel to the center axis and passes the edge of the exit surface 2.

In the case where the width d1 is determined based on the boundary B1 and the position P2, the width d1 is expressed as the distance Δ2 between the boundary B1 and the position P2. The position P2 is the point of intersection of the inner light distribution surface 3a and specific illumination light. The specific illumination light is the illumination light that passes the position farthest from the position P1 among the illumination light incident on the observation area.

Next, the third example of the inner light distribution surface will be described with reference to FIG. 9C. In the case of the third example of the inner light distribution surface, the inner light distribution surface 51a abuts on the outer light distribution surface 51b and a surface S3.

The inner light distribution surface 51a includes a first inner surface 51a1 and a second inner surface 51a2. The first inner surface 51a1 abuts on the outer light distribution surface 51b. The second inner surface 51a2 abuts on the surface S3.

The first inner surface 51a1 is a curved surface. The outer light distribution surface 3b is a flat surface, and therefore the boundary B1 of the first inner surface 51a1 and the outer light distribution surface 51b is distinct. The second inner surface 51a2 is a flat surface. The surface S3 is a flat surface as with the second inner surface 51a2, and therefore the boundary of the second inner surface 51a2 and the flat surface S3 is not distinct. For this reason, the width d1 cannot be determined based on the boundaries, in the case of the third example of the inner light distribution surface. In the case of the third example of the inner light distribution surface, the width d1 is determined based on the boundary B1 and the position P3 or on the boundary B1 and the position P4.

In the case where the width d1 is determined based on the boundary B1 and the position P3, the width d1 is expressed as the distance Δ3 between the boundary B1 and the position P3. In the case where the width d1 is determined based on the boundary B1 and the position P4, the width d1 is expressed as the distance Δ4 between the boundary B1 and the position P4. The position P3 is the point of intersection of the inner light distribution surface 51a and the straight line SL. The position P4 is the point of intersection of the inner light distribution surface 51a and the specific illumination light.

In the case of the first example, the boundary B2 may be regarded as the point of intersection of the inner light distribution surface 3a and the specific illumination light. Alternatively, the boundary B2 may be located closer to the center axis than the point of intersection of the inner light distribution surface 3a and the specific illumination light.

If conditional expression (1) is satisfied, it is possible to prevent a decrease in the illumination efficiency while achieving a wide light distribution.

If the value of d1/d2 falls below the lower limit value of conditional expression (1), the width of the outer light distribution surface is too large. Then, the width of the inner light distribution surface is relatively small. The inner light distribution surface causes illumination light to converge and then diverge. The small width of the inner light distribution surface leads to small divergence of illumination light. This makes the light distribution narrow.

If value of d1/d2 exceeds the upper limit value of conditional expression (1), the width of the outer light distribution surface is too small. This makes the quantity of illumination light that is cast outside the observation area large. This leads to a decrease in the illumination efficiency.

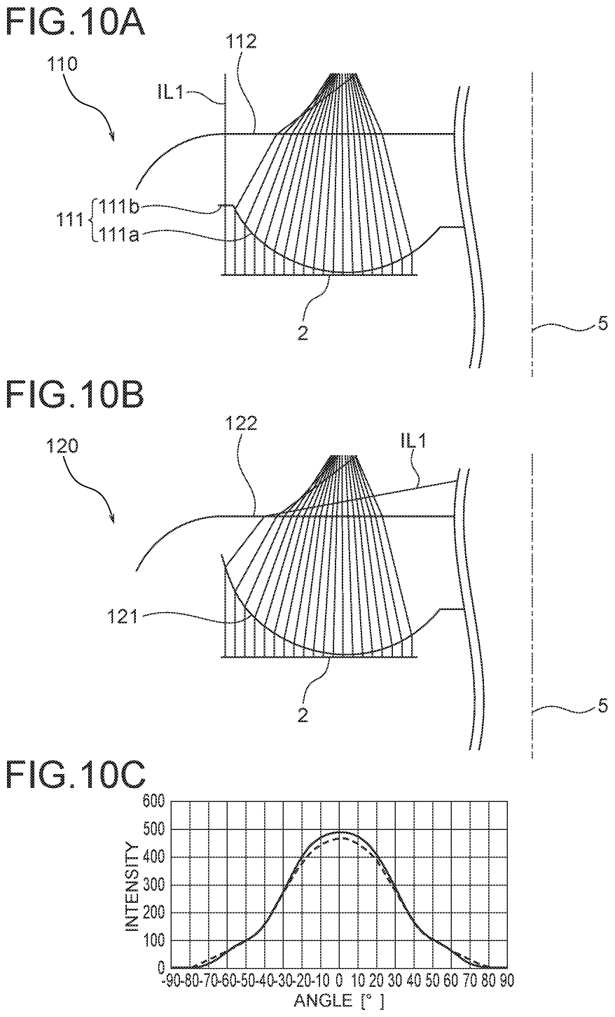

FIGS. 10A, 10B, and 10C show endoscope illumination systems and their light distributions. FIG. 10A is a diagram showing a sixth exemplary endoscope illumination system. FIG. 10B is a diagram showing a conventional endoscope illumination system. FIG. 10C is a graph showing light distributions of illumination light.

While illumination light goes out from the exit surface in various directions, FIGS. 10A and 10B show only illumination light that goes out from the exit surface parallel to the center axis.

The endoscope illumination system 110 will be described with reference to FIG. 10A. As described above, the endoscope illumination system 110 is the sixth example of the endoscope illumination system according to the embodiment.

The endoscope illumination system 110 includes an exit surface 2, an entrance side optical surface 111, and an exit side optical surface 112. In the endoscope illumination system 110, illumination light is emitted from the exit surface 2.

The illumination light is incident on the entrance side optical surface 111. The entrance side optical surface 111 includes an inner light distribution surface 111a and an outer light distribution surface 111b. The outer light distribution surface 111b is located farther from the center axis 5 than the inner light distribution surface 111a.

The inner light distribution surface 111a includes a first inner surface. The first inner surface is a curved surface that is convex to the exit surface 2. In the case shown in FIG. 10A, the inner light distribution surface 111a is constituted only by a curved surface that is convex to the exit surface 2, that is, the inner light distribution surface 111a is constituted only by the first inner surface.

The outer light distribution surface in the endoscope illumination system of this embodiment is a flat surface or a curved surface that is concave to the exit surface 2. In the case of the endoscope illumination system 110, the outer light distribution surface 111b is a flat surface.

The illumination light IL1 passes through the outer light distribution surface 111b and the exit side optical surface 112. The illumination light IL1 is incident on a flat portion of the outer light distribution surface 111b and a flat portion of the exit side optical surface 112. Therefore, the illumination light IL1 travels parallel to the center axis 5.

The value of d1/d2 in the endoscope illumination system 110 is 20.4. Thus, the sixth exemplary endoscope illumination system satisfies conditional expression (1).

Another endoscope illumination system 120 will be described with reference to FIG. 10B. The endoscope illumination system 120 shown in FIG. 10B is a conventional endoscope illumination system. This endoscope illumination system 120 has an exit surface 2, an entrance side optical surface 121, and an exit side optical surface 122. The entrance side optical surface 121 is constituted only by a curved surface that is convex to the exit surface 2.

In the endoscope illumination system 120, the illumination light IL1 goes out from the exit side optical surface 122. However, the illumination light IL 2 is refracted so largely on the entrance side optical surface 121 that the illumination light IL 2 is cast outside the observation area of the endoscope. This leads to a decrease in the illumination efficiency.

In contrast, in the case of the endoscope illumination system 110, the illumination light IL1 is not refracted on the entrance side optical surface 111 nor the exit side optical surface 122 but travels parallel to the center axis 5. Therefore, the illumination light IL1 is not cast outside the observation area of the endoscope. In consequence, it is possible to prevent a decrease in the illumination efficiency.

In FIG. 10C, the solid curve represents the distribution of illumination light with the endoscope illumination system 110, and the broken curve represents the distribution of illumination light with the endoscope illumination system. 120. FIG. 10C shows the light distributions in the case where the endoscope illumination systems are arranged symmetrically with respect to the center axis 5. The horizontal axis represents the angle, and the vertical axis represents the intensity.

In the case of the endoscope illumination system. 110, the angle at which the intensity becomes zero is smaller than 80°. In contrast, in the case of the endoscope illumination system 120, the angle at which the intensity becomes zero is approximately 80°. Since it is considered that the range of the angle represents the extent of the illumination area, FIG. 10C indicates that the illumination area of the endoscope illumination system 110 is smaller than the illumination area of the endoscope illumination system 60.

The smaller illumination area means a smaller quantity of illumination light that is cast outside the observation area. Therefore, the endoscope illumination system 110 can illuminate the observation area more efficiently than the endoscope illumination system 120.

As described above, the outer light distribution surface 111b is located farther from the center axis 5 than the inner light distribution surface 111a. In the case where the center of the observation area is located on the center axis 5, the illumination light IL1 is incident on the peripheral portion of the observation area. Thus, the peripheral portion of the observation area can be illuminated brightly.

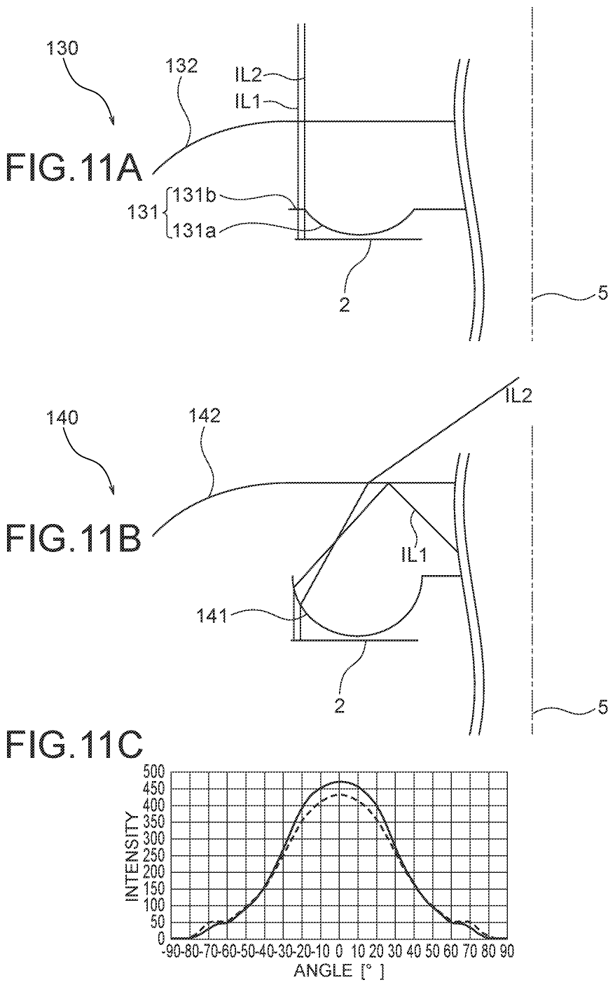

FIGS. 11A, 11B, and 11C show endoscope illumination systems and their light distributions. FIG. 11A is a diagram showing a seventh exemplary endoscope illumination system. FIG. 11B is a diagram showing a conventional endoscope illumination system. FIG. 11C is a graph showing light distributions of illumination light.

While illumination light goes out from the exit surface in various directions, FIGS. 11A and 11B show only illumination light that goes out from the exit surface parallel to the center axis.

The endoscope illumination system 130 will be described with reference to FIG. 11A. As described above, the endoscope illumination system. 130 is the seventh example of the endoscope illumination system according to the embodiment.

The endoscope illumination system 130 includes an exit surface 2, an entrance side optical surface 131, and an exit side optical surface 132. In the endoscope illumination system 130, illumination light is emitted from the exit surface 2.

The illumination light is incident on the entrance side optical surface 131. The entrance side optical surface 131 includes an inner light distribution surface 131a and an outer light distribution surface 131b. The outer light distribution surface 131b is located farther from the center axis 5 than the inner light distribution surface 131a.

The inner light distribution surface 131a includes a first inner surface. The first inner surface is a curved surface that is convex to the exit surface 2. In the case shown in FIG. 11A, the inner light distribution surface 131a is constituted only by a curved surface that is convex to the exit surface 2, that is, the inner light distribution surface 131a is constituted only by the first inner surface.

The outer light distribution surface in the endoscope illumination system of this embodiment is a flat surface or a curved surface that is concave to the exit surface. In the case of the endoscope illumination system 130, the outer light distribution surface 131b is a flat surface.

The illumination light IL1 and the illumination light IL2 pass through the outer light distribution surface 131b and the exit side optical surface 132. The illumination light IL1 and the illumination light IL2 are incident on a flat portion of the outer light distribution surface 131b and a flat portion of the exit side optical surface 132. Therefore, the illumination light IL1 and the illumination light IL2 travel parallel to the center axis 5.

The value of d1/d2 in the endoscope illumination system 130 is 16.9. Thus, the seventh exemplary endoscope illumination system satisfies conditional expression (1).

Another endoscope illumination system 140 will be described with reference to FIG. 11B. The endoscope illumination system 140 shown in FIG. 11B is a conventional endoscope illumination system. This endoscope illumination system 140 has an exit surface 2, an entrance side optical surface 141, and an exit side optical surface 142. The entrance side optical surface 141 is constituted only by a curved surface that is convex to the exit surface 2.

In the case of this endoscope illumination system 140, the illumination light IL1 does not go out from the exit side optical surface 142. The illumination light IL2 goes out from the exit side optical surface 142. However, the illumination light IL 2 is refracted so largely on the entrance side optical surface 141 that the illumination light IL 2 is cast outside the observation area of the endoscope. This leads to a decrease in the illumination efficiency.

In contrast, in the case of the endoscope illumination system 130, the illumination light IL1 and the illumination light IL2 are not refracted on the entrance side optical surface 131 nor the exit side optical surface 132 but travel parallel to the center axis 5. In consequence, the illumination light IL1 and the illumination light IL 2 are not cast outside the observation area. Therefore, it is possible to prevent a decrease in the illumination efficiency.

In FIG. 11C, the solid curve represents the distribution of illumination light with the endoscope illumination system 130, and the broken curve represents the distribution of illumination light with the endoscope illumination system. 140. FIG. 11C shows the light distributions in the case where the endoscope illumination systems are arranged symmetrically with respect to the center axis 5. The horizontal axis represents the angle, and the vertical axis represents the intensity.

In the case of the endoscope illumination system. 130, the angle at which the intensity becomes zero is smaller than 80°. In contrast, in the case of the endoscope illumination system 140, the angle at which the intensity becomes zero is approximately 80°. Since it is considered that the range of the angle represents the extent of the illumination area, FIG. 11C indicates that the illumination area of the endoscope illumination system 130 is smaller than the illumination area of the endoscope illumination system 140.

The smaller illumination area means a smaller quantity of illumination light that is cast outside the observation area. Therefore, the endoscope illumination system 130 can illuminate the observation area more efficiently than the endoscope illumination system 140.

As described above, the outer light distribution surface 131b is located farther from the center axis 5 than the inner light distribution surface 131a. In the case where the center of the observation area is located on the center axis 5, the illumination light IL1 and the illumination light IL2 are incident on the peripheral portion of the observation area. Thus, the peripheral portion of the observation area can be illuminated brightly.

The actual values of the term “d1/d2” defined in conditional expression (1) in the respective exemplary endoscope illumination systems are presented below. All the exemplary endoscope illumination systems satisfy conditional expression (1).

| | d1/d2 |
|---|---|
| First Example | 11.1 |
| Second Example | 8.1 |
| Third Example | 14.1 |
| Fifth Example | 8 |
| Sixth Example | 20.4 |
| Seventh Example | 16.9 |

It is preferred that the endoscope illumination system according to the embodiment include a light transmissive member, an inner surface of the light transmissive member include the entrance side optical surface, and an outer surface of the light transmissive member include the exit side optical surface.

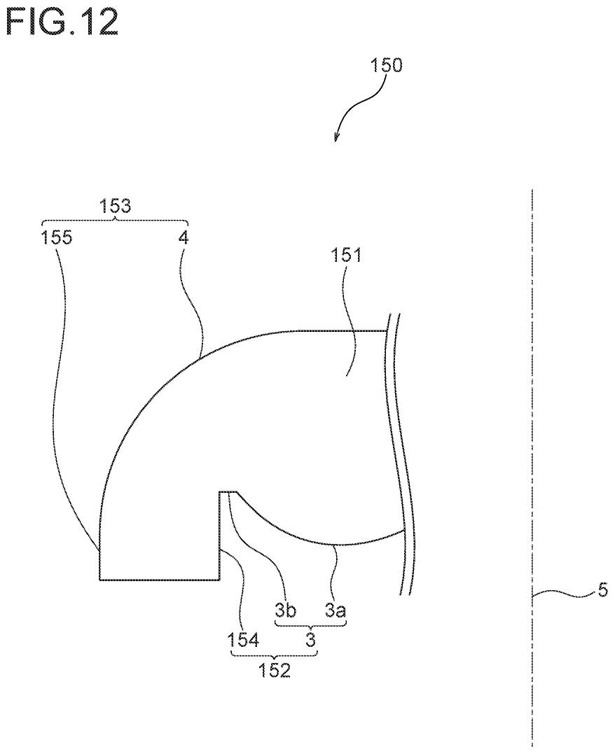

FIG. 12 is a diagram showing an endoscope illumination system according to the embodiment. FIG. 12 shows an eighth example of the endoscope illumination system according to the embodiment. The elements same as those in FIG. 1A are denoted by the same reference numerals and will not be described further.

The eighth exemplary illumination system uses one light transmissive member. The endoscope illumination system 150 includes a light transmissive member 151. The light transmissive member has an inner surface 152 and an outer surface 153.

The inner surface 152 includes the entrance side optical surface 3 and an inner circumferential surface 154. The outer surface 153 includes the exit side optical surface 4 and an outer circumferential surface 155.

The light transmissive member 151 may be used as an end cover. The light transmissive member 151 may be made by molding. In the case where the outer light distribution surface 3b is absent, the inner light distribution surface 3a abuts directly on the inner circumferential surface 154. In that case, a flat portion may be formed between the inner circumferential surface 154 and the inner light distribution surface 3a unintentionally in the process of molding.

The light transmissive member 151 has the outer light distribution surface 3b between the inner light distribution surface 3a and the inner circumferential surface 154. The outer light distribution surface 3b is a surface that is intentionally provided, and it is not a surface that is formed unintentionally.

It is preferred that the endoscope illumination system according to the embodiment include a first light transmissive member and a second light transmissive member, the first light transmissive member be located between the exit surface and the second light transmissive member, an inner surface of the first light transmissive member include the entrance side optical surface, and an outer surface of the second light transmissive member include the exit side optical surface.

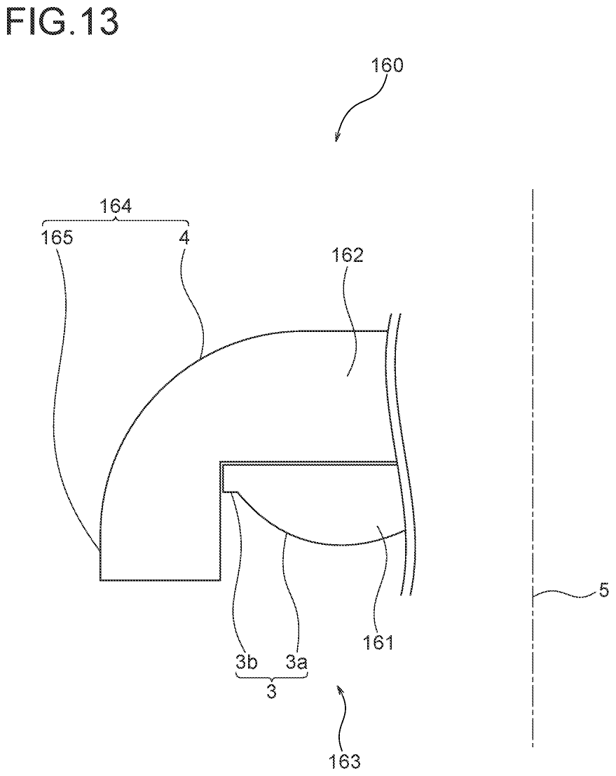

FIG. 13 is a diagram showing another endoscope illumination system according to the embodiment. FIG. 13 shows a ninth example of the endoscope illumination system according to the embodiment. The elements same as those in FIG. 1A are denoted by the same reference numerals and will not be described further.

The ninth exemplary endoscope illumination system uses two light transmissive members. The endoscope illumination system 160 shown in FIG. 13 includes a first light transmissive member 161 and a second light transmissive member 162. The first light transmissive member 161 is located between the exit surface and the second light transmissive member 162.

The first light transmissive member 161 has an inner surface 163. The inner surface 163 includes the entrance side optical surface 3.

The second light transmissive member 162 has an outer surface 164. The outer surface 164 includes the exit side optical surface 4 and an outer circumferential surface 165.

It is preferred that the first light transmissive member 161 be in contact with the second light transmissive member 162, though a gap between the first light transmissive member 161 and the second light transmissive member 162 is depicted in FIG. 13 to facilitate understanding.

It is preferred that the endoscope illumination system according to the embodiment include a first region and a second region that are formed by dividing the insert part into two regions by an imaginary plane containing the center axis, and the exit surface, the entrance side optical surface, and the exit side optical surface be provided in each of the first and the second regions.

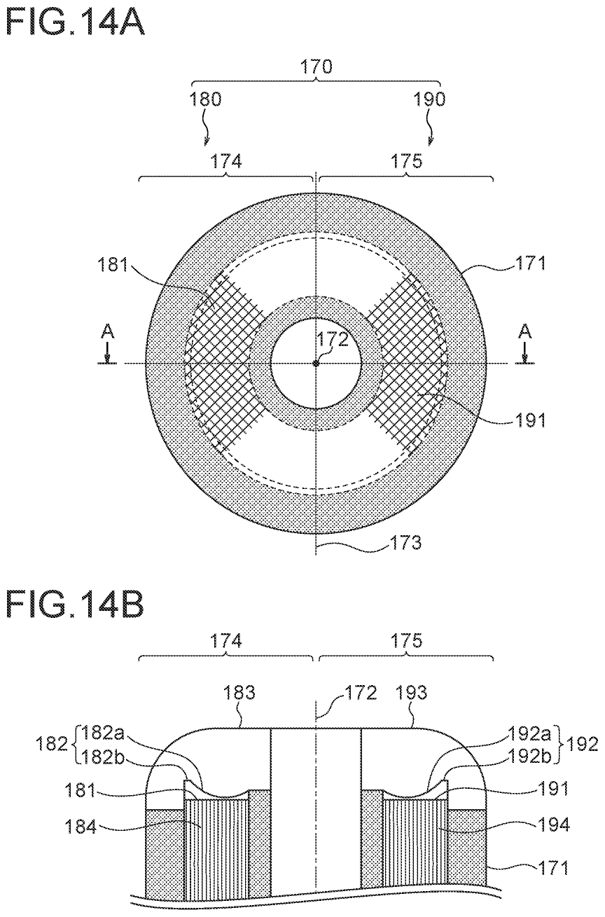

FIGS. 14A, 14B, and 14C are diagrams illustrating an endoscope illumination system according to the embodiment. FIG. 14A is a front view of the end of the insert part. FIG. 14B is a cross sectional view of the endo portion of the insert part taken along sectional line A-A.

The endoscope illumination system 170 will be described. The endoscope illumination system 170 is a tenth example of the endoscope illumination system according to the embodiment. The endoscope illumination system 170 is provided in the insert part 171.

The insert part 171 can be divided into two regions by an imaginary plane containing the center axis 172. The straight line 173 indicates the position of the imaginary plane. One of the two regions will be referred to as the first region 174, and the other will be referred to as the second region 175.

The endoscope illumination system 170 includes an endoscope illumination system 180 provided in the first region 174 and an endoscope illumination system 190 provided in the second region 175.

The endoscope illumination system 180 includes an exit surface 181, an entrance side optical surface 182, and an exit side optical surface 183. The exit surface 181 is the end face of alight guide 184. Illumination light emitted from the exit surface 181 is incident on the entrance side optical surface 182.

The entrance side optical surface 182 includes an inner light distribution surface 182a and an outer light distribution surface 182b. The outer light distribution surface 182b is located farther from the center axis 172 than the inner light distribution surface 182a. The center axis 172 is the center axis of the insert part 171.

The inner light distribution surface 182a includes a first inner surface. The first inner surface is a curved surface that is convex to the exit surface 181. In the system shown in FIG. 14B, the inner light distribution surface 182a is constituted only by a curved surface that is convex to the exit surface 181. Thus, the inner light distribution surface 182a is constituted only by the first inner surface. The outer light distribution surface 182b is a flat surface.

The endoscope illumination system 190 includes an exit surface 191, an entrance side optical surface 192, and an exit side optical surface 193. The exit surface 191 is the end face of a light guide 194. Illumination light emitted from the exit surface 191 is incident on the entrance side optical surface 192.

The entrance side optical surface 192 includes an inner light distribution surface 192a and an outer light distribution surface 192b. The outer light distribution surface 192b is located farther from the center axis 172 than the inner light distribution surface 192a.

The inner light distribution surface 192a includes a first inner surface. The first inner surface is a curved surface that is convex to the exit surface 191. In the system shown in FIG. 14B, the inner light distribution surface 192a is constituted only by a curved surface that is convex to the exit surface 191. Thus, the inner light distribution surface 192a is constituted only by the first inner surface. The outer light distribution surface 192b is a flat surface.

In the system shown in FIG. 14A, the shapes of the exit surface 181 and the exit surface 191 are partial circles. Alternatively, the shape of the exit surface may be a circle, an ellipse, a polygon, or a comb-shape (i.e. a rectangle with one side replaced by a partial circle).

It is preferred in the endoscope illumination system of this embodiment that the exit surface, the entrance side optical surface, and the exit side optical surface in the first region be the same as those in the second region.

The two endoscope illumination systems 180 and 190 in the endoscope illumination system 170 are the same. The shape of the exit surface 191 is the same as the shape of the exit surface 181. The shape of the entrance side optical surface 192 is the same as the shape of the entrance side optical surface 182. The shape of the exit side optical surface 193 is the same as the shape of the exit side optical surface 183.

It is preferred in the endoscope illumination system according to the embodiment that the entrance side optical surface in the first region and that in the second region be different from each other.

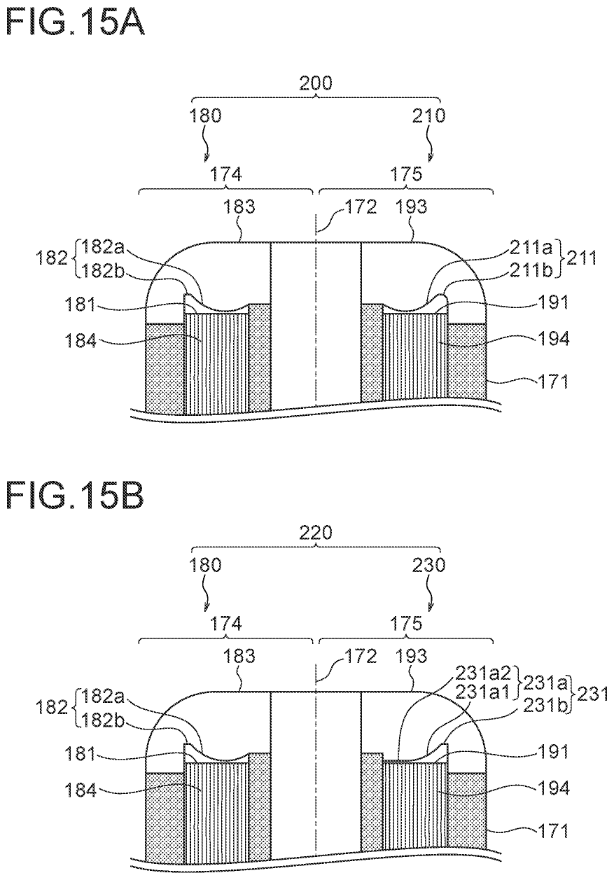

FIGS. 15A and 15B show endoscope illumination systems according to the embodiment. FIG. 15A is a diagram showing an eleventh exemplary endoscope illumination system. FIG. 15B is a diagram showing a twelfth exemplary endoscope illumination system. The elements same as those in FIG. 14B are denoted by the same reference numerals and will not be described further.

The endoscope illumination system 200 will be described with reference to FIG. 15A. The endoscope illumination system 200 is the eleventh example of the endoscope illumination system according to the embodiment. The endoscope illumination system 200 is provided in the insert part 171.

The endoscope illumination system 200 includes endoscope illumination systems 180 and 210. The endoscope illumination system 210 is provided in the second region 175.

The endoscope illumination system 210 includes an exit surface 191, an entrance side optical surface 211, and an exit side optical surface 193. Illumination light emitted from the exit surface 191 is incident on the entrance side optical surface 211.

The entrance side optical surface 211 includes an inner light distribution surface 211a and an outer light distribution surface 211b. The outer light distribution surface 211b is located farther from the center axis 172 than the inner light distribution surface 211a.

The inner light distribution surface 211a includes a first inner surface. The first inner surface is a curved surface that is convex to the exit surface 191. In the system shown in FIG. 15A, the inner light distribution surface 211a is constituted only by a curved surface that is convex to the exit surface 191. Thus, the inner light distribution surface 211a is constituted only by the first inner surface. The outer light distribution surface 211b is a curved surface that is concave to the exit surface 191.

The two endoscope illumination systems 180 and 210 in the endoscope illumination system 200 are not the same. The shape of the entrance side optical surface 211 is different from the shape of the entrance side optical surface 182. Specifically, while the outer light distribution surface 182b in the endoscope illumination system 180 is a flat surface, the outer light distribution surface 211b in the endoscope illumination system 210 is a curved surface.

The endoscope illumination system 220 will be described with reference to FIG. 15B. The endoscope illumination system 220 is the twelfth example of the endoscope illumination system according to the embodiment. The endoscope illumination system 220 is provided in the insert part 171.

The endoscope illumination system 220 includes endoscope illumination systems 180 and 230. The endoscope illumination system 230 is provided in the second region 175.

The endoscope illumination system 230 includes an exit surface 191, an entrance side optical surface 231, and an exit side optical surface 193. Illumination light emitted from the exit surface 191 is incident on the entrance side optical surface 231.

The entrance side optical surface 231 includes an inner light distribution surface 231a and an outer light distribution surface 231b. The outer light distribution surface 231b is located farther from the center axis 172 than the inner light distribution surface 231a.

The inner light distribution surface 231a includes a first inner surface 231a1 and a second inner surface 231a2. The first inner surface 231a1 is a curved surface that is convex to the exit surface 191. The second inner surface 231a2 is a flat surface. The outer light distribution surface 231b is a flat surface.

The two endoscope illumination systems 180 and 230 in the endoscope illumination system 220 are not the same. The shape of the entrance side optical surface 231 is different from the shape of the entrance side optical surface 182. Specifically, while the inner light distribution surface 182b in the endoscope illumination system 180 is constituted only by a curved surface, the inner light distribution surface 231a in the endoscope illumination system 230 is constituted by a flat surface and a curved surface.

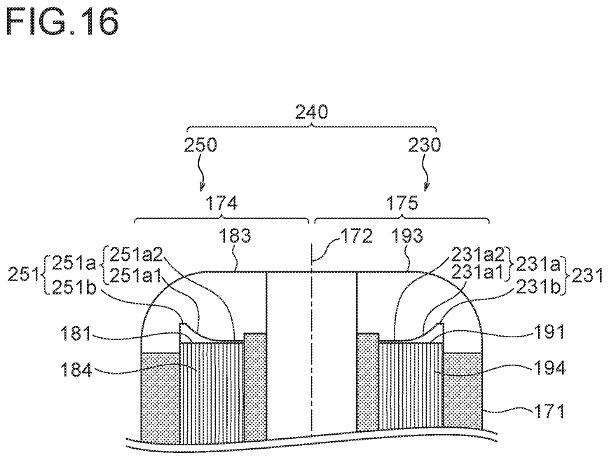

FIG. 16 shows an endoscope illumination system according to the embodiment. FIG. 16 is a diagram showing a thirteenth exemplary endoscope illumination system. The elements same as those in FIG. 15B are denoted by the same reference numerals and will not be described further.

The endoscope illumination system 240 will be described with reference to FIG. 16. The endoscope illumination system 240 is the thirteenth example of the endoscope illumination system according to the embodiment. The endoscope illumination system 240 is provided in the insert part 171.

The endoscope illumination system 240 includes endoscope illumination systems 230 and 250. The endoscope illumination system 250 is provided in the first region 174.

The endoscope illumination system 250 includes an exit surface 181, an entrance side optical surface 251, and an exit side optical surface 183. Illumination light emitted from the exit surface 181 is incident on the entrance side optical surface 251.

The entrance side optical surface 251 includes an inner light distribution surface 251a and an outer light distribution surface 251b. The outer light distribution surface 251b is located farther from the center axis 172 than the inner light distribution surface 251a.

The inner light distribution surface 251a includes a first inner surface 251a1 and a second inner surface 251a2. The first inner surface 251a1 is a curved surface that is convex to the exit surface 181. The second inner surface 251a2 is a flat surface. The outer light distribution surface 251b is a flat surface.

The two endoscope illumination systems 240 and 250 in the endoscope illumination system 240 are not the same. The width of the first inner surface 251a1 is different from the width of the first inner surface 231a1. The width of the second inner surface 251a2 is different from the width of the second inner surface 231a2.

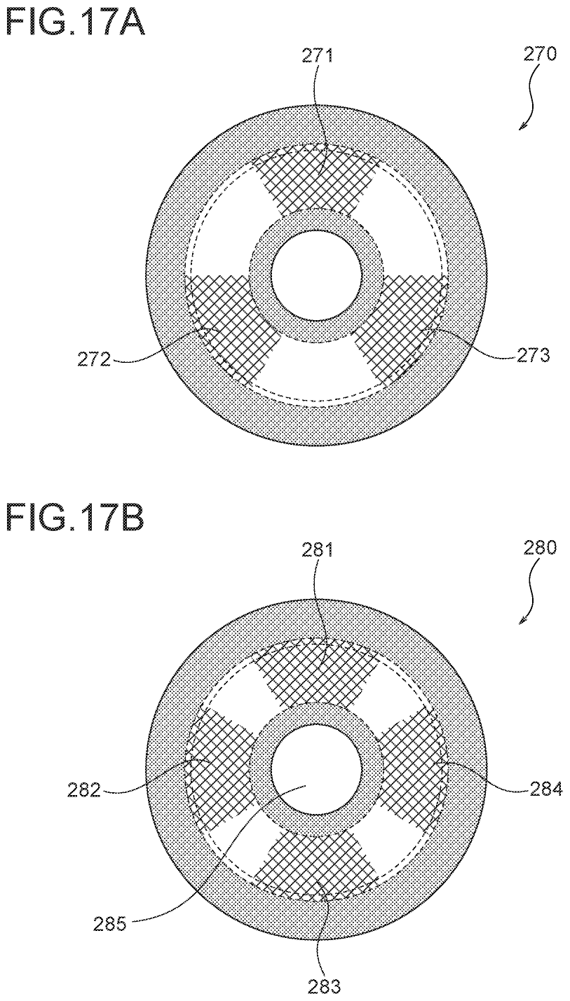

FIGS. 17A and 17B show endoscope illumination systems according to the embodiment. FIG. 17A is a diagram showing a fourteenth exemplary endoscope illumination system. FIG. 17B is a diagram showing a fifteenth exemplary endoscope illumination system.

The endoscope illumination system 270 will be described with reference to FIG. 17A. The endoscope illumination system 270 is the fourteenth example of the endoscope illumination system according to the embodiment. This endoscope illumination system 270 includes exit surfaces 271, 272, and 273.

Entrance side optical surfaces and exit side optical surfaces are provided at positions opposed to the respective exit surfaces 271, 272, and 273.

The endoscope illumination system 270 is provided with three exit surfaces. Accordingly, the number of the entrance side optical surfaces and the number of the exit side optical surfaces are also three. One exit surface may be the same as or different from another exit surface. One entrance side optical surface may be the same as or different from another entrance side optical surface.

The endoscope illumination system 280 will be described with reference to FIG. 17B. The endoscope illumination system 280 is the fifteenth example of the endoscope illumination system according to the embodiment. The endoscope illumination system 280 includes exit surfaces 281, 282, 283, and 284.

Entrance side optical surfaces and exit side optical surfaces are provided at positions opposed to the respective exit surfaces 281, 282, 283, and 284.

The endoscope illumination system 280 is provided with four surfaces. Accordingly, the number of the entrance side optical surfaces and the number of the exit side optical surfaces are also four. One exit surface may be the same as or different from another exit surface. One entrance side optical surface may be the same as or different from another entrance side optical surface.

The insert part has a cylindrical space 285 at its center. An objective optical system or other components may be provided in this space 285. In the case of the fifteenth exemplary endoscope illumination system, the center axis of the space 285 coincides with the center axis of the insert part. Therefore, if an objective optical system is provided in this space 285, the objective optical system is not eccentric to the center of the insert part.

It is preferred in the endoscope illumination system according to the embodiment that the exit surface in the first region be different from the exit surface in the second region.

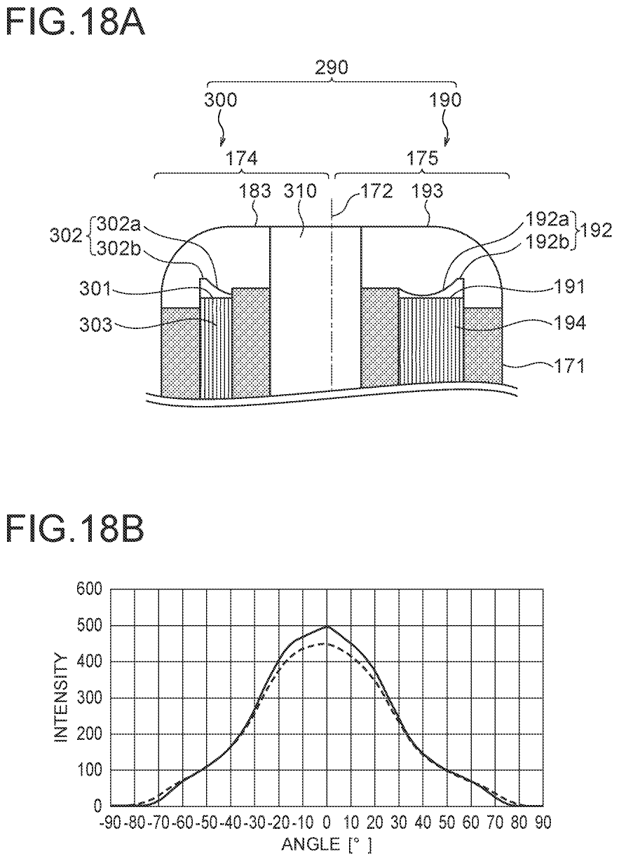

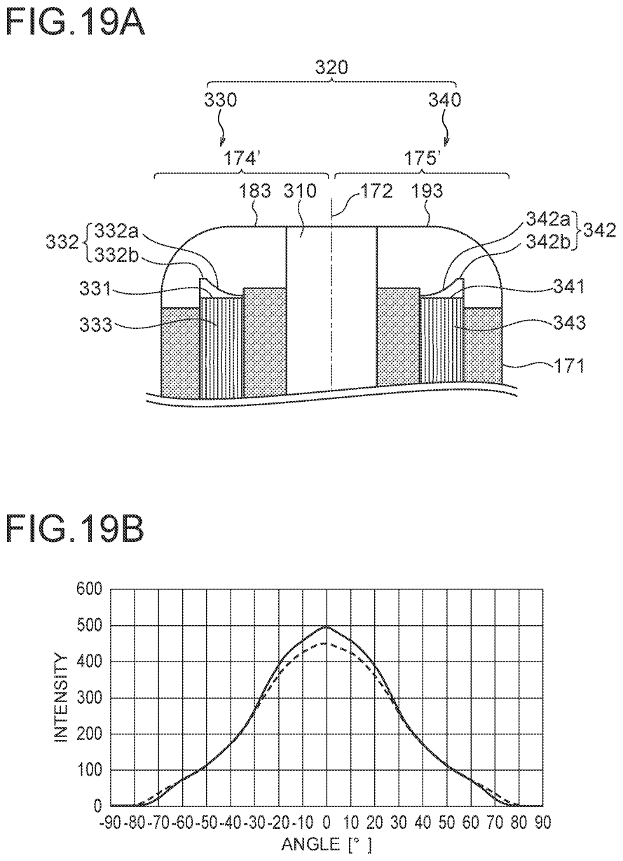

FIGS. 18A, 18B, 19A, 19B show an endoscope illumination system according to the embodiment and its light distribution. FIGS. 18A and 19A are diagrams showing a sixteenth exemplary endoscope illumination system. FIGS. 18B and 19B are graphs showing light distributions of illumination light. The elements same as those in FIG. 14B are denoted by the same reference numerals and will not be described further.

The sixteenth exemplary endoscope illumination system includes four endoscope illumination systems as with the endoscope illumination system 280 shown in FIG. 17B. As described above, in the case of the endoscope illumination system 280, the center axis of the space 285 coincides with the center axis of the insert part. In contrast, in the case of the sixteenth exemplary endoscope illumination system, the center axis of the cylindrical space is decentered from the center axis of the insert part.

In the endoscope illumination system 280, the direction going from the endoscope illumination system 283 to the endoscope illumination system 281 will be referred to as the first direction, and the direction going from the endoscope illumination system 282 to the endoscope illumination system 284 will be referred to as the second direction. The cylindrical space in the sixteenth exemplary endoscope illumination system is decentered in the first direction but not decentered in the second direction.

The sixteenth exemplary endoscope illumination system includes an endoscope illumination system 290 arranged along the first direction and an endoscope illumination system 320 arranged along the second direction.

The endoscope illumination system 290 will be described with reference to FIG. 18A. The endoscope illumination system 290 is provided in the insert part 171. The endoscope illumination system 290 includes endoscope illumination systems 300 and 190. The endoscope illumination system 300 is located in the first region 174.

The endoscope illumination system 300 includes an exit surface 301, an entrance side optical surface 302, and an exit side optical surface 183. The exit surface 301 is the end face of alight guide 303. Illumination light emitted from the exit surface 301 is incident on the entrance side optical surface 302.

The entrance side optical surface 302 includes an inner light distribution surface 302a and an outer light distribution surface 302b. The outer light distribution surface 302b is located farther from the center axis 172 than the inner light distribution surface 302a.

The inner light distribution surface 302a includes a first inner surface. The first inner surface is a curved surface that is convex to the exit surface 301. The inner light distribution surface 302a shown in FIG. 18A is constituted only by a curved surface that is convex to the exit surface 301. Thus, the inner light distribution surface 302a is constituted only by the first inner surface. The outer light distribution surface 302b is a flat surface.

The center axis of the cylindrical space 310 is decentered from the center axis 172 along the first direction. More than one half of the space 310 is situated in the first region 174. In this case, the space that can accommodate an endoscope illumination system is smaller in the first region 174 than in the second region 175.

For this reason, the endoscope illumination systems 300 and 190 in the endoscope illumination system 290 are not the same. The width of the exit surface 301 is different from the width of the exit surface 191. Specifically, the width of the exit surface 301 is smaller than the width of the exit surface 191. The shape of the entrance side optical surface 302 is different from the shape of the entrance side optical surface 192. The width of the entrance side optical surface 302 is smaller than the width of the entrance side optical surface 192.

In FIG. 18B, the solid curve represents the distribution of illumination light with the endoscope illumination system 290, and the broken curve represents the distribution of illumination light with a conventional endoscope illumination system (not shown). The horizontal axis represents the angle, and the vertical axis represents the intensity.

In the case of the endos cope illumination system 290, the angle at which the intensity becomes zero is smaller than 80°. In contrast, in the case of the conventional endoscope illumination system, the angle at which the intensity becomes zero is approximately 80°. Since it is considered that the range of the angle represents the extent of the illumination area, FIG. 18B indicates that the illumination area of the endoscope illumination system 290 is smaller than the illumination area of the conventional endoscope illumination system.

The smaller illumination area means a smaller quantity of illumination light that is cast outside the observation area. Therefore, the endoscope illumination system 290 can illuminate the observation area more efficiently than the conventional endoscope illumination system.

The endoscope illumination system 320 will be described with reference to FIG. 19A. The endoscope illumination system 320 is provided in the insert part 171. The endoscope illumination system 320 includes endoscope illumination systems 330 and 340. The endoscope illumination system 330 is located in a third region 174′. The endoscope illumination system 340 is located in a fourth region 175′.

The third and fourth regions are regions that are formed by dividing the insert part into two regions by another imaginary plane. This imaginary plane is a plane containing a straight line perpendicular to the straight line 173 in FIG. 14A.

The endoscope illumination system 330 includes an exit surface 331, an entrance side optical surface 332, and an exit side optical surface 183. The exit surface 331 is the end face of alight guide 333. Illumination light emitted from the exit surface 331 is incident on the entrance side optical surface 332.

The entrance side optical surface 332 includes an inner light distribution surface 332a and an outer light distribution surface 332b. The outer light distribution surface 332b is located farther from the center axis 172 than the inner light distribution surface 332a.

The inner light distribution surface 332a includes a first inner surface. The first inner surface is a curved surface that is convex to the exit surface 331. The inner light distribution surface 332a shown in FIG. 19A is constituted only by a curved surface that is convex to the exit surface 331. Thus, the inner light distribution surface 332a is constituted only by the first inner surface. The outer light distribution surface 332b is a flat surface.

The endoscope illumination system 340 includes an exit surface 341, an entrance side optical surface 342, and an exit side optical surface 193. The exit surface 341 is the end face of alight guide 343. Illumination light emitted from the exit surface 341 is incident on the entrance side optical surface 342.

The entrance side optical surface 342 includes an inner light distribution surface 342a and an outer light distribution surface 342b. The outer light distribution surface 342b is located farther from the center axis 172 than the inner light distribution surface 342a.

The inner light distribution surface 342a includes a first inner surface. The first inner surface is a curved surface that is convex to the exit surface 341. The inner light distribution surface 342a shown in FIG. 19A is constituted only by a curved surface that is convex to the exit surface 341. Thus, the inner light distribution surface 342a is constituted only by the first inner surface. The outer light distribution surface 342b is a flat surface.

The center axis of the cylindrical space 310 is not decentered from the center axis 172 along the second direction. One half of the space 310 is situated in the first region 174′ and the other half is situated in the second region 175′. In this case, the space that can accommodate an endoscope illumination system is the same in the first region 174′ and in the second region 175′.

For this reason, the endoscope illumination systems 330 and 340 in the endoscope illumination system 320 are the same. The shape of the exit surface 341 is the same as the shape of the exit surface 331. The shape of the entrance side optical surface 342 is the same as the shape of the entrance side optical surface 332. The shape of the exit side optical surface 193 is the same as the shape of the exit side optical surface 183.

In FIG. 19B, the solid curve represents the distribution of illumination light with the endoscope illumination system 320, and the broken curve represents the distribution of illumination light with a conventional endoscope illumination system (not shown). The horizontal axis represents the angle, and the vertical axis represents the intensity.

In the case of the endoscope illumination system. 320, the angle at which the intensity becomes zero is smaller than 80°. In contrast, in the case of the conventional endoscope illumination system, the angle at which the intensity becomes zero is approximately 80°. Since it is considered that the range of the angle represents the extent of the illumination area, FIG. 19B indicates that the illumination area of the endoscope illumination system 320 is smaller than the illumination area of the conventional endoscope illumination system.

The smaller illumination area means a smaller quantity of illumination light that is cast outside the observation area. Therefore, the endoscope illumination system 320 can illuminate the observation area more efficiently than the conventional endoscope illumination system.

The endoscope illumination system according to this embodiment includes exit surfaces, the entrance side optical surfaces, and the exit side optical surfaces provided not only in the first and second regions but also in the third and fourth regions. The third region and the fourth region are two regions that are formed by dividing the insert part by an imaginary plane that is perpendicular to the imaginary plane that partitions the first region and the second region. The entrance side optical surface in the second region is larger than the entrance side optical surface in the first region and satisfies conditional expression (2) below; and the entrance side optical surface in the third region is the same as the entrance side optical surface in the fourth region and satisfies conditional expression (3) below:

8≤din2/dout2≤32 (2)

8≤din3/dout3≤26 (3)

where din2 is the width of the inner light distribution surface in the second region, dout2 is the width of the outer light distribution surface in the second region, din3 is the width of the inner light distribution surface in the third region, and dout3 is the width of the outer light distribution surface in the third region.

As described above, in the sixteenth exemplary endoscope illumination system, the entrance side optical surface 192 in the second region 175 is larger than the entrance side optical surface 302 in the first region 174. The entrance side optical surface 332 in the third region 174′ is the same as the entrance side optical surface 332 in the fourth region 175′.

The actual values of the terms defined in conditional expressions (2) and (3) in the sixteenth exemplary endoscope illumination system are presented below. For the first and fourth regions also, the ratio of the width of the inner light distribution surface (din1, din4) and the width of the outer light distribution surface (dout1, dout4) is given below.

| | | |
|---|---|---|
| First region | din1/dout1 | 17.5 |
| Second region | din2/dout2 | 31.5 |
| Third region | din3/dout3 | 25.1 |
| Fourth region | din4/dout4 | 25.1 |

The sixteenth exemplary endoscope illumination system satisfies conditional expressions (2) and (3). Satisfying conditional expressions (2) and (3) can ensure wide light distribution and prevent a decrease in the illumination efficiency.

In the cylindrical space 310 may be provided an objective optical system or other components. The center axis of the cylindrical space 310 is decentered from the center axis 172 along the first direction. Therefore, if an objective optical system is provided in this space 310, the objective optical system is decentered from the center of the insert part. In spite of that, the sixteenth exemplary endoscope illumination system can prevent a decrease in the illumination efficiency while achieving wide light distribution, because it satisfies conditional expressions (2) and (3).

An endoscope according to the embodiment includes the endoscope illumination system according to the above-described embodiment and an objective optical system. The endoscope illumination system is located farther from the center axis than the objective optical system.

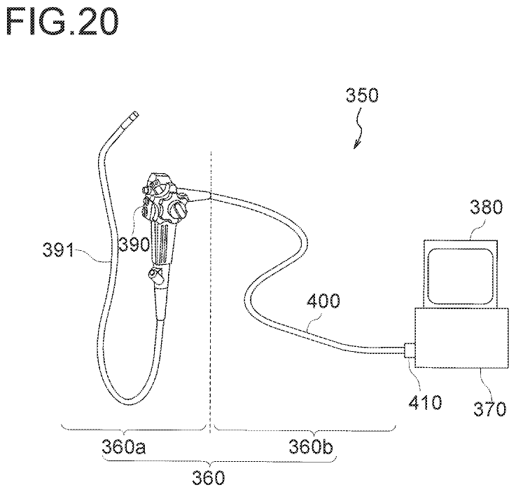

FIG. 20 is a diagram showing an endoscope system. In the illustration of the system in FIG. 20, an endoscope alone is illustrated in an enlarged manner to show some details of its structure.

The endoscope system 350 includes an endoscope 360 and an image processing apparatus 370. The endoscope 360 includes a scope part 360a and a connection cord part 360b. The image processing apparatus 370 is connected with a display unit 380.

The scope part 360a includes an operation unit 390 and an insert part 391. The insert part 391 is thin and long and can be inserted into the body cavity of a patient. The insert part 391 is made of a flexible member. The observer can perform various operations using an angle knob and/or other parts provided in the operation unit 390.

The connection cord part 360b extends from the operation unit 390. The connection cord part 360b includes a universal cord 400. The universal cord 400 is connected to the image processing apparatus 370 by a connector 410.

The universal cord 400 is used to transmit various signals. The signals transmitted include a power voltage signal and a CCD driving signal. These signals are sent from a power source apparatus or a video processor to the scope part 360a. The signals transmitted also include an image signal. This signal is sent from the scope part 360a to the video processor.

The video processor, which is included in the image processing apparatus 370, may be connected with peripheral devices, such as a video printer (not shown). The video processor applies signal processing on the image signal sent from the scope part 360a. The display unit 380 displays an endoscope image based on the image signal on its display screen.

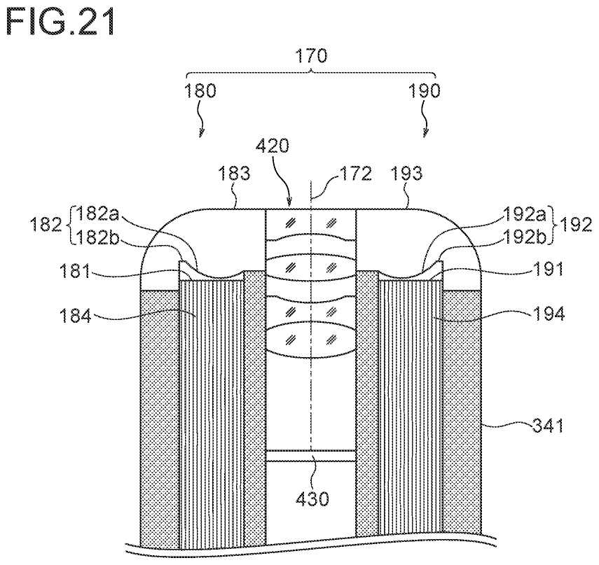

FIG. 21 is a cross sectional view of the end portion of the insert part. The components same as those in FIGS. 14A and 14B are denoted by the same reference numerals and will not be described further.

In the end portion of the insert part 391 are provided the endoscope illumination system 170 and an objective optical system 420. The endoscope illumination system 170 is located farther from the center axis 172 than the objective optical system 420.

The objective optical system 420 forms an image of an object. The image of the object is picked up by an imager 430. Thus, the image of the object can be obtained.

In the system shown in FIG. 21, the objective optical system 420 is not decentered from the center axis 172. In consequence, the optical axis of the objective optical system 420 coincides with the center axis 172. However, the objective optical system 420 may be decentered from the center axis 172.

The endoscope illumination system according to the embodiment is one that is provided in the insert part and includes an exit surface from which illumination light is emitted, an entrance side optical surface on which the illumination light is incident, and an exit side optical surface from which the illumination light goes out. The entrance side optical surface includes, in order away from the center axis of the insert part, a first flat surface, a first curved surface abutting on the first flat surface, and a second flat surface abutting on the first curved surface. The exit side optical surface includes, in order away from the center axis of the insert part, a third flat surface, and a second curved surface abutting on the third flat surface. The first curved surface is a surface that is convex to the exit surface, and the second curved surface is a surface that is convex to the outside.

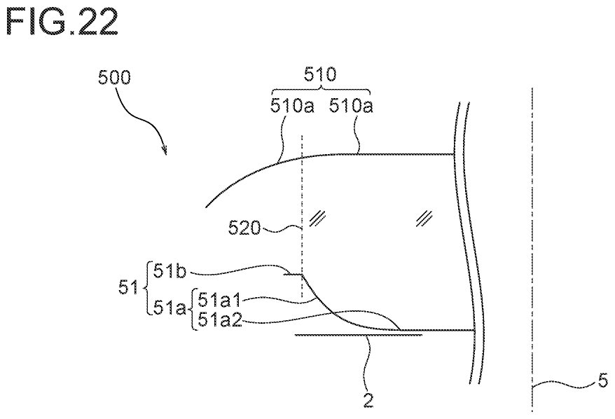

FIG. 22 is a diagram showing an endoscope illumination system according to the embodiment. FIG. 22 is a diagram showing a seventeenth exemplary endoscope illumination system. The elements same as those in FIG. 5 are denoted by the same reference numerals and will not be described further.

The endoscope illumination system 500 is the seventeenth exemplary endoscope illumination system. This endoscope illumination system 500 includes an exit surface 2, an entrance side optical surface 51, and an exit side optical surface 510.

The entrance side optical surface 51 includes a second inner surface 51a2 as a first flat surface, a first inner surface 51a1 as a first curved surface, and an outer light distribution surface 51b as a second flat surface. Thus, the entrance side optical surface 51 includes, in order away from the center axis 5 of the insert part, the first flat surface, the first curved surface abutting on the first flat surface, and the second flat surface abutting on the first curved surface. The first curved surface is a surface that is convex to the exit surface 2.

The exit side optical surface 510 includes a first exit side surface 510a and a second exit side surface 510b. The first exit side surface 510a is a flat surface. The second exits side surface 510b is a curved surface.

The first exit side surface 510a is a third flat surface and the second exit side surface 510b is a second curved surface. Thus, the exit side optical surface 510 includes, in order away from the center axis 5 of the insert part, the third flat surface, and the second curved surface abutting on the third flat surface. The second curved surface is a surface that is convex to the outside.

It is preferred in the endoscope illumination system according to the embodiment that a straight line that is parallel to the center axis and passes the boundary of the first curved surface and the second flat surface intersect with the second curved surface.

FIG. 22 shows a straight line 520. The straight line 520 is a line that is parallel to the center axis and passes the boundary of the first curved surface and the second flat surface. In the endoscope illumination system 500, the straight line 520 intersects with the second exit side surface 510b, or the second curved surface.

It is preferred in the endoscope illumination system according to the embodiment that the proportion of the rays that pass through the first curved surface and the third flat surface be equal to or larger than 70% of all the rays emitted from the exit surface.

The rays emitted from the exit surface pass through the entrance side optical surface and the exit side optical surface. As described above, the entrance side optical surface includes the first flat surface, the first curved surface and the second flat surface, and the exit side optical surface includes the third flat surface and the second curved surface. The rays emitted perpendicularly from the exit surface can be separated into first to fourth ray groups.

The first ray group is made up of rays that pass through the first flat surface and the third flat surface. The second ray group is made up of rays that pass through the first curved surface and the third flat surface. The third ray group is made up of rays that pass through the first curved surface and the second curved surface. The fourth ray group is made up of rays that pass through the second flat surface and the second curved surface.

Curved surfaces are apt to be affected by manufacturing errors. Hence, the relative position and angle of the first curved surface and the second curved surface are likely to vary. The third ray group is made up of rays that pass through the first curved surface and the second curved surface. The larger the quantity of light of the third ray group is, the larger the variation in the light distribution due to manufacturing errors tends to be.

To achieve a wide light distribution, the first curved surface is necessary. The rays that pass through the second curved surface can be reduced by increasing the rays that pass through the third flat surface. The second ray group is made up of rays that pass through the first curved surface and the third flat surface. By making the proportion of the second ray group equal to or larger than 70% of all the rays passing through the first curved surface, it is possible to achieve an illumination design that tends not to be affected by manufacturing errors.

It is preferred in the endoscope illumination system according to the embodiment that the exit surface include a first exit surface located in the first region and a second exit surface located in the second region and the following conditional expression (4) be satisfied:

120°≤α+β (4)

where α is the central angle of a sector region that includes the first exit surface, β is the central angle of a sector region that includes the second exit surface, the sector region is a region defined by two radii of a specific circle and the arc between the radii, the specific circle is a circle in an imaginary plane perpendicular to the center axis, and the center of the specific circle is located on the center axis.

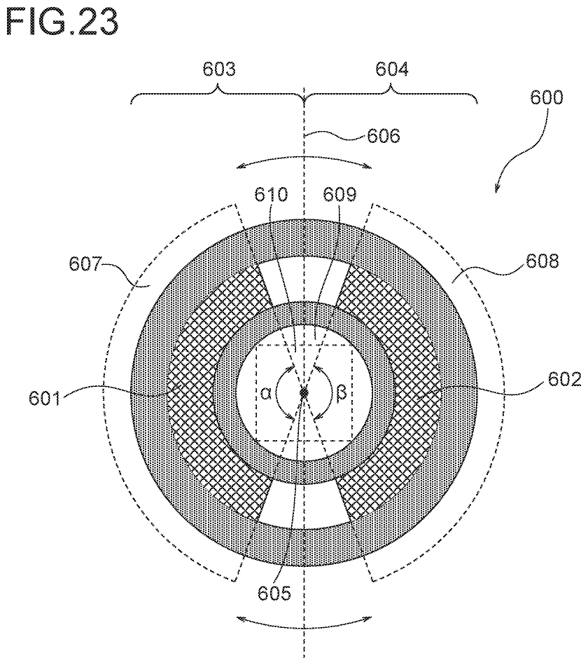

FIG. 23 illustrates an endoscope illumination system according to the embodiment. FIG. 23 is a diagram showing an eighteenth exemplary endoscope illumination system.

The endoscope illumination system 600 shown in FIG. 23 is the eighteenth example of the endoscope illumination system according to the embodiment. The exit surface of the endoscope illumination system 600 includes a first exit surface 601 and a second exit surface 602. The first exit surface is located in a first region 603, and the second exit surface 602 is located in a second region 604. The first region 603 and the second region 604 are two regions partitioned by an imaginary plane containing the center axis 605. The straight line 606 in FIG. 23 indicates the position of the imaginary plane.

The first exit surface 601 is included in a sector region 607. The second exit surface 602 is included in a sector region 608. Each of the sector regions 607 and 608 is a region defined by two radii of a specific circle and the arc between the radii. The specific circle is a circle in an imaginary plane perpendicular to the center axis 605, and the center of the specific circle is located on the center axis 605.

The light emitted from the first exit surface 601 of the endoscope illumination system 600 travels toward the second region 604. The light emitted from the second exit surface 602 travels toward the first region 603.

The larger the first exit surface 601 is, the larger the quantity of light that travels toward the second region 604 is. The larger the second exit surface 602 is, the larger the quantity of light that travels toward the first region 603 is. In consequence, the illumination area in front can be illuminated widely and brightly.

The angle α is the central angle of the sector region 607. The larger the first exit surface 601 is, the larger the angle α is. The angle β is the central angle of the sector region 608. The larger the second exit surface 602 is, the larger the angle β is.

If conditional expression (4) is satisfied, the first exit surface 601 and the second exit surface 602 can be made sufficiently large. In consequence, the illumination area can be illuminated widely and brightly. In the case shown in FIG. 23, α+β=220°.

If conditional expression (4) is not satisfied, the first exit surface 601 and the second exit surface 602 cannot be made sufficiently large. Then, the regions indicated by the arrows in FIG. 23 are large. Illumination light does not reach the regions indicated by the arrows, or the quantity of light that reaches the regions indicated by the arrows is small. Hence, the illumination area cannot be illuminated widely and brightly.

It is preferred that the following conditional expression (4′) be satisfied instead of conditional expression (4).

180°≤α+β (4′)

If conditional expression (4′) is satisfied, the illumination area can be illuminated more widely and brightly.

As shown in FIG. 21, an objective optical system and an imager are provided in the space 609. FIG. 23 shows a light receiving surface 610 of the imager. The larger the angle α and the angle β are, the larger the quantity of light that travels in the diagonal direction of the light receiving surface 610 is.

As described above, if conditional expression (4) is satisfied, the illumination area in front can be illuminated widely and brightly. Hence, the object can be imaged excellently.

In the configuration shown in FIG. 23, the space 609 is not decentered from the center axis 605. However, the space 609 may be decentered from the center axis 609, as with the endoscope illumination system 290 shown in FIG. 18A. Even in the case where the space 609 is decentered, if conditional expression (4) is satisfied, the illumination area in front can be illuminated widely and brightly. Hence, the object can be imaged satisfactorily.

It is preferred in the endoscope illumination system according to the embodiment that the width of the second inner surface be varied depending on the location.

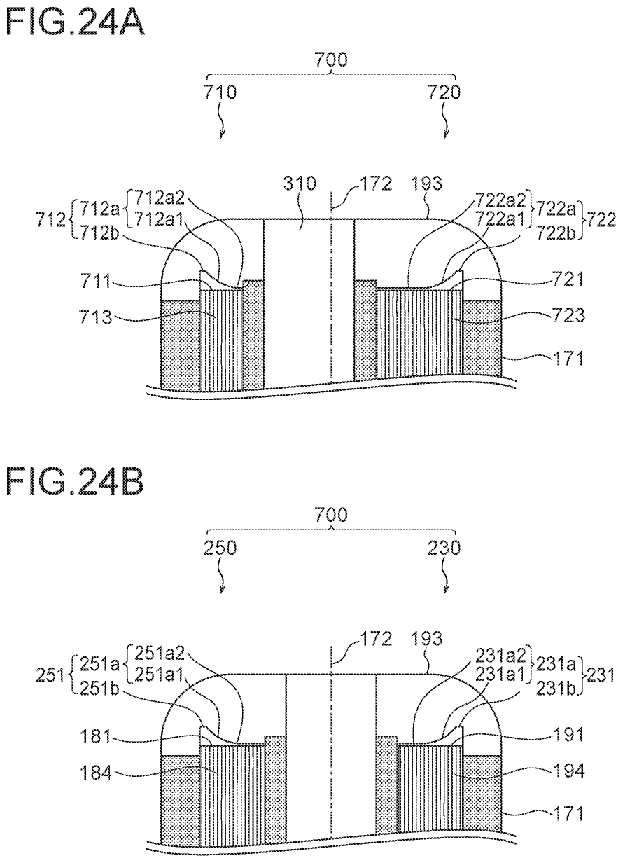

FIGS. 24A and 24B are diagrams showing an endoscope illumination system according to the embodiment. FIG. 24 is a diagram showing a nineteenth exemplary endoscope illumination system. FIG. 24A shows the endoscope illumination system along the direction in which the system is decentered. FIG. 24B shows the endoscope illumination system along the direction in which the system is not decentered. The elements same as those in FIG. 16 are denoted by the same reference numerals and will not be described further.

The endoscope illumination system 700 includes endoscope illumination systems 230, 250, 710, and 720. The objective optical system of the endoscope illumination system 700 is decentered from the center of the insert part.

As described above, in the endoscope illumination system 280 shown in FIG. 17B, the first direction is defined as the direction from the endoscope illumination system 283 toward the endoscope illumination system 281. The second direction is defined as the direction from the endoscope illumination system 282 toward the endoscope illumination system 284. The cylindrical space in the nineteenth exemplary endoscope illumination system is decentered along the first direction but not decentered along the second direction.

The endoscope illumination system 710 includes an exit surface 711, an entrance side optical surface 712, and an exit side optical surface 193. The exit surface 711 is the end face of a light guide 713. Illumination light emitted from the exit surface 711 is incident on the entrance side optical surface 712.

The entrance side optical surface 712 includes an inner light distribution surface 712a and an outer light distribution surface 712b. The outer light distribution surface 712b is located farther from the center axis 172 than the inner light distribution surface 712a.

The inner light distribution surface 712a includes a first inner surface 712a1 and a second inner surface 712a2. The first inner surface 712a1 is a curved surface that is convex to the exit surface 711. The second inner surface 712a2 is a flat surface. The outer light distribution surface 712b is a flat surface.

The endoscope illumination system 720 includes an exit surface 721, an entrance side optical surface 722, and an exit side optical surface 193. The exit surface 721 is the end face of a light guide 723. Illumination light emitted from the exit surface 721 is incident on the entrance side optical surface 722.

The entrance side optical surface 722 includes an inner light distribution surface 722a and an outer light distribution surface 722b. The outer light distribution surface 722b is located farther from the center axis 172 than the inner light distribution surface 722a.

The inner light distribution surface 722a includes a first inner surface 722a1 and a second inner surface 722a2. The first inner surface 722a1 is a curved surface that is convex to the exit surface 721. The second inner surface 722a2 is a flat surface. The outer light distribution surface 722b is a flat surface.

The center axis of the cylindrical space 310 is decentered from the center axis 172 in the first direction. More than one half of the space 310 is situated in the side of the endoscope illumination system 710. In this case, the space that can accommodate an endoscope illumination system is smaller in the side of the endoscope illumination system 710 than in the side of the endoscope illumination system 720.

Therefore, the endoscope illumination systems 710 and 720 in the endoscope illumination system. 700 are not the same. The width of the exit surface 711 is different from the width of the exit surface 721. Specifically, the width of the exit surface 711 is smaller than the width of the exit surface 721.

The shape of the entrance side optical surface 712 is different from the shape of the entrance side optical surface 722. The width of the entrance side optical surface 712 is smaller than the width of the entrance side optical system 722. The width of the second inner surface 712a2 is smaller than the width of the second inner surface 722a2.

In the case where the objective optical system is decentered from the center of the insert part, in order to achieve uniformity in light distribution, it is desirable that the positional relationship of the curved surface in the entrance side optical surface and the curved surface in the exit side optical surface be always the same irrespective of their positions. In order to maintain the positional relationship of the curved surface in the entrance side optical surface and the curved surface in the exit side optical surface, the width of the second inner surface may be varied depending on the position. This can reduce non-uniformity in light distribution.

The exit surface of the endoscope illumination system according to the embodiment may include either one surface or a plurality of surfaces. For example, in the case where the shape of the exit surface is a circle, the exit surface includes one surface. In the case where the shape of the exit surface is a partial circle, the exit surface includes a plurality of surfaces. The partial circle is a shape of a portion cut out from a circle.

The entrance side optical surface may include either one surface or a plurality of surfaces. For example, in the case where the shape of the entrance side optical surface is a circle, the exit surface includes one surface. In the case where the shape of the entrance side optical surface is a partial circle, the exit surface includes a plurality of surfaces.

As above, the present invention is suitable for an endoscope illumination system having high illumination efficiency and an endoscope provided with such an endoscope illumination system.

The present invention can provide an endoscope illumination system having high illumination efficiency and an endoscope provided with such an endos cope illumination system.

What is claimed is:

1. An endoscope illumination system provided in an insert part, comprising:
an exit surface from which illumination light is emitted;
an entrance side optical surface on which the illumination light is incident; and
an exit side optical surface from which the illumination light goes out,
wherein the entrance side optical surface includes an inner light distribution surface and an outer light distribution surface,
the outer light distribution surface is located farther from the center axis of the insert part than the inner light distribution surface,
the inner light distribution surface includes a first inner surface,
the first inner surface is a curved surface that is convex to the exit surface, and
the outer light distribution surface is a flat surface or a curved surface that is concave to the exit surface.

2. An endoscope illumination system according to claim 1, wherein the inner light distribution surface includes the first inner surface and a second inner surface, the second inner surface is a flat surface, and the second inner surface is located closer to the center axis than the first inner surface.

3. An endoscope illumination system according to claim 1, wherein the exit side optical surface includes a first exit side surface and a second exist side surface, the first exit side surface is a flat surface, the second exit side surface is a curved surface, the second exit side surface is located farther from the center axis than the first exit side surface, and a straight line that is parallel to the center axis and passes the boundary of the first exit side surface and the second exit side surface intersects with the exit surface.

4. An endoscope illumination system according to claim 1, wherein the endoscope illumination system satisfies the following conditional expression (1):
8≤d1/d2≤32 (1)
where d1 is the width of the inner light distribution surface, and d2 is the width of the outer light distribution surface.

5. An endoscope illumination system according to claim 1, comprising a light transmissive member, wherein an inner surface of the light transmissive member includes the entrance side optical surface, and an outer surface of the light transmissive member includes the exit side optical surface.

6. An endoscope illumination system according to claim 1, comprising a first light transmissive member and a second light transmissive member, wherein the first light transmissive member is located between the exit surface and the second light transmissive member, an inner surface of the first light transmissive member includes the entrance side optical surface, an outer surface of the second light transmissive member includes the exit side optical surface.

7. An endoscope illumination system according to claim 1, wherein a first region and a second region are defined as two regions formed by dividing the insert part by an imaginary plane containing the center axis, and the exit surface, the entrance side optical surface, and the exit side optical surface are provided in each of the first and second regions.

8. An endoscope illumination system according to claim 7, wherein the exit surface, the entrance side optical surface, and the exit side optical surface are the same between the first region and the second regions.

9. An endoscope illumination system according to claim 7, wherein the entrance side optical surface is different between the first region and the second regions.

10. An endoscope illumination system according to claim 7, wherein the exit surface is different between the first region and the second regions.

11. An endoscope illumination system according to claim 7, wherein a third region and a fourth region are defined as two regions formed by dividing the insert part by another imaginary plane perpendicular to said imaginary plane,
the exit surface, the entrance side optical surface, and the exit side optical surface are provided in each of the third and fourth regions,
the entrance side optical surface in the second region is larger than the entrance side optical surface in the first region and satisfies the following conditional expression (2), and
the entrance side optical surface in the third region is the same as the entrance side optical surface in the fourth region and satisfies the following conditional expression (3):
8≤din2/dout2≤32 (2)
8≤din3/dout3≤26 (3)
where din2 is the width of the inner light distribution surface in the second region, dout2 is the width of the outer light distribution surface in the second region, din3 is the width of the inner light distribution surface in the third region, and dout3 is the width of the outer light distribution surface in the third region.

12. An endoscope illumination system provided in an insert part, comprising:
an exit surface from which illumination light is emitted;
an entrance side optical surface on which the illumination light is incident; and
an exit side optical surface from which the illumination light goes out,
wherein the entrance side optical surface includes, in order away from the center axis of the insert part, a first flat surface, a first curved surface abutting on the first flat surface, and a second flat surface abutting on the first curved surface,
the exit side optical surface includes, in order away from the center axis of the insert part, a third flat surface and a second curved surface abutting on the third flat surface,
the first curved surface is a surface convex to the exit surface, and
the second curved surface is a surface convex to the outside.

13. An endoscope illumination system according to claim 12, wherein a straight line that is parallel to the center axis and passes through the boundary of the first curved surface and the second flat surface intersects with the second curved surface.

14. An endoscope comprising:
an endoscope illumination system according to claim 1; and
an objective optical system,
wherein the endoscope illumination system is located farther from the center axis than the objective optical system.

15. An endoscope comprising:
an endoscope illumination system according to claim 12; and
an objective optical system,
wherein the endoscope illumination system is located farther from the center axis than the objective optical system.
